SEQUENCE LISTING (75 sequences)

SEQ ID NO: 1 - DNA, 26 bp, Artificial sequence
  PCR primer
ggtcgtttttcttcattccttagtcg

SEQ ID NO: 2 - DNA, 24 bp, Artificial sequence
  PCR primer
tctcttcatcgctgctcatcctcc

SEQ ID NO: 3 - DNA, 22 bp, Artificial sequence
  PCR primer
tccccttcccattttccactcg

SEQ ID NO: 4 - DNA, 26 bp, Artificial sequence
  PCR primer
gcacttacacagacagctcctccacc

SEQ ID NO: 5 - DNA, 22 bp, Artificial sequence
  PCR primer
caggaggaaccagagcccagtc

SEQ ID NO: 6 - DNA, 23 bp, Artificial sequence
  PCR primer
tggagtagggcagggaggacagt

SEQ ID NO: 7 - DNA, 27 bp, Artificial sequence
  PCR primer
ggctgggaagtttggtgatatagtgtc

SEQ ID NO: 8 - DNA, 24 bp, Artificial sequence
  PCR primer
agcagccaaatccttcagtctcaa

SEQ ID NO: 9 - DNA, 28 bp, Artificial sequence
  PCR primer
aagacaaaaactcccccattgtgaaata

SEQ ID NO: 10 - DNA, 28 bp, Artificial sequence
  PCR primer
gccctcctgaaaatgtgtaaagaaatgt

SEQ ID NO: 11 - DNA, 25 bp, Artificial sequence
  PCR primer
cttcccccactcccttcaaacttac

SEQ ID NO: 12 - DNA, 20 bp, Artificial sequence
  PCR primer
agcacttgacggcagcagca

SEQ ID NO: 13 - DNA, 22 bp, Artificial sequence
  PCR primer
tgccccgagacctgataaccaa

SEQ ID NO: 14 - DNA, 25 bp, Artificial sequence
  PCR primer
tcagaacaagctggaggcaactagg

SEQ ID NO: 15 - DNA, 27 bp, Artificial sequence
  PCR primer
ggaatagaaagcgactcactcaccagg

SEQ ID NO: 16 - DNA, 25 bp, Artificial sequence
  PCR primer
ccacttttagctgagtgcctgtccc

SEQ ID NO: 17 - DNA, 25 bp, Artificial sequence
  PCR primer
ctgtctctgcaatgatgaaatggcc

SEQ ID NO: 18 - DNA, 21 bp, Artificial sequence
  PCR primer
ggaaactctgggcatgggcag

SEQ ID NO: 19 - DNA, 22 bp, Artificial sequence
  PCR primer
actgggcaggagattcggttat

SEQ ID NO: 20 - DNA, 25 bp, Artificial sequence
  PCR primer
cgccccagattaactgatagttgct

SEQ ID NO: 21 - DNA, 24 bp, Artificial sequence
  PCR primer
atactaagggcaggtgaggctcca

SEQ ID NO: 22 - DNA, 24 bp, Artificial sequence
  probe
tcgtttttcttcattccttagtcg

SEQ ID NO: 23 - DNA, 25 bp, Artificial sequence
  probe
atgaaacaagaccaaagactcactg

SEQ ID NO: 24 - DNA, 19 bp, Artificial sequence
  probe
tgacccagcttgacagcca

SEQ ID NO: 25 - DNA, 22 bp, Artificial sequence
  probe
ggcaatcgctgaagacagaaag

SEQ ID NO: 26 - DNA, 22 bp, Artificial sequence
  probe
gagaacaggtgtaagtgccgcc

SEQ ID NO: 27 - DNA, 24 bp, Artificial sequence
  probe
ttggattcacggttaagagagttc

SEQ ID NO: 28 - DNA, 24 bp, Artificial sequence
  probe
tccagtcccaaaggttaatttctc

SEQ ID NO: 29 - DNA, 21 bp, Artificial sequence
  probe
ccgaagttgagtgcatacccg

SEQ ID NO: 30 - DNA, 25 bp, Artificial sequence
  probe
caaaatcaaggatggctagaaacac

SEQ ID NO: 31 - DNA, 23 bp, Artificial sequence
  probe
cttccaaagtatagcctccccag

SEQ ID NO: 32 - DNA, 22 bp, Artificial sequence
  probe
ggtgagtttgtttcctcctccc

SEQ ID NO: 33 - DNA, 24 bp, Artificial sequence
  probe
cccaaaagtaagtgctctcctgcc

SEQ ID NO: 34 - DNA, 22 bp, Artificial sequence
  probe
cggggagaagtggaaactctgg

SEQ ID NO: 35 - DNA, 24 bp, Artificial sequence
  probe
tcagagttattccttttccaaatg

SEQ ID NO: 36 - DNA, 25 bp, Artificial sequence
  probe
cgccccagattaactgatagttgct

SEQ ID NO: 37 - DNA, 19 bp, Artificial sequence
  probe
ggtccctgctccaaactgc

SEQ ID NO: 38 - DNA, 50 bp, Artificial sequence
  sequence amplified by PCR
cttgagagatgctgctgtgtactactgcatcctgagagacgggggggggg

SEQ ID NO: 39 - DNA, 65 bp, Artificial sequence
  sequence amplified by PCR
ccttttgaggagctccagatgaaagactctgcctcttacctctgtgctgtgaggaatgggggggg

SEQ ID NO: 40 - DNA, 41 bp, Artificial sequence
  sequence amplified by PCR
gcgatgtatttctgtgcttacatgagcccgggggggggggg

SEQ ID NO: 41 - DNA, 328 bp, Artificial sequence
  sequence amplified by PCR
tattctgtatctgatgatgtctttgagaacaggtgtaagtgccgccaaaaatgaagtggagcagagtcctcagaacctgactgcccaggaaggagaatttatcacaatcaactgcagttactcggtaggaataagtgccttacactggctgcaacagcatccaggaggaggcattgtttccttgtttatgctgagctcagggaagaagaagcatggaagattaattgccacaataaacatacaggaaaagcacagctccctgcacatcacagcctcccatcccagagactctgccgtctacatctgtgctgtcagagggggggggggg

SEQ ID NO: 42 - DNA, 44 bp, Artificial sequence
  sequence amplified by PCR
actcagccgtgtactactgtcttctgggagatgggggggggggg

SEQ ID NO: 43 - DNA, 162 bp, Artificial sequence
  sequence amplified by PCR
acctggagcaggtctccagcttgctgacgtatattttttcaaatatggacatgaaacaagaccaaagactcactgttctattgaataaaaaggataacatctgtctctgcgcattgcagacacccagactggggactcagctatctacttctgtgcagagag

SEQ ID NO: 44 - DNA, 215 bp, Artificial sequence
  sequence amplified by PCR
actactcatcgtctgtttcacngtatctcttctggtatgtgcaataccccaaccaaggactccagcttctcctgaagtannnatcaggnnccaccctggttaaaggcatcaacggttttgaggctgaatttaacaagagtgaaacntccttccacntgangaaaccctcagcccatatnagcgacncggctgagtacttctgtgctgtgagtgat

SEQ ID NO: 45 - DNA, 163 bp, Artificial sequence
  sequence amplified by PCR
acctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagagagt

SEQ ID NO: 46 - DNA, 24 bp, Artificial sequence
  PCR primer
gcaacatgctggcggagcacccac

SEQ ID NO: 47 - DNA, 22 bp, Artificial sequence
  PCR primer
atggctttgcagagcactctgg

SEQ ID NO: 48 - DNA, 19 bp, Artificial sequence
  PCR primer
gcctctgcacccatctcga

SEQ ID NO: 49 - DNA, 24 bp, Artificial sequence
  PCR primer
gaggatgtggagcagagtcttttc

SEQ ID NO: 50 - DNA, 24 bp, Artificial sequence
  PCR primer
cggccaccctgacctgcaactata

SEQ ID NO: 51 - DNA, 25 bp, Artificial sequence
  PCR primer
gggaccccagcagggagacgttgcc

SEQ ID NO: 52 - DNA, 24 bp, Artificial sequence
  PCR primer
atgctcctgttgctcataccagtg

SEQ ID NO: 53 - DNA, 24 bp, Artificial sequence
  PCR primer
cctgaaagccacgaaggctgatga

SEQ ID NO: 54 - DNA, 21 bp, Artificial sequence
  PCR primer
gcatctgacgaccttcttggt

SEQ ID NO: 55 - DNA, 24 bp, Artificial sequence
  PCR primer
ccatgatgcggggactggagttgc

SEQ ID NO: 56 - DNA, 24 bp, Artificial sequence
  PCR primer
cattcgttcaaatgtgggcgaaaa

SEQ ID NO: 57 - DNA, 24 bp, Artificial sequence
  PCR primer
cagaagataactcaaacccaacca

SEQ ID NO: 58 - DNA, 21 bp, Artificial sequence
  PCR primer
agagtgactcagcccgagaag

SEQ ID NO: 59 - DNA, 24 bp, Artificial sequence
  PCR primer
ccgggcagcagacactgcttctta

SEQ ID NO: 60 - DNA, 24 bp, Artificial sequence
  PCR primer
tcgtcggaactcttttgatgagca

SEQ ID NO: 61 - DNA, 21 bp, Artificial sequence
  PCR primer
gtcttgtggcttcagcttggc

SEQ ID NO: 62 - DNA, 24 bp, Artificial sequence
  PCR primer
tgcctcgctggataaatcatcagg

SEQ ID NO: 63 - DNA, 24 bp, Artificial sequence
  PCR primer
gggagctctgctggggctcttgag

SEQ ID NO: 64 - DNA, 20 bp, Artificial sequence
  PCR primer
gcagcttcccttccagcaat

SEQ ID NO: 65 - DNA, 24 bp, Artificial sequence
  PCR primer
ggagaggacttcaccacgtactgc

SEQ ID NO: 66 - DNA, 21 bp, Artificial sequence
  PCR primer
ggctggtggcaagagtaactg

SEQ ID NO: 67 - DNA, 24 bp, Artificial sequence
  PCR primer
cactgcggcccagcctggtgatac

SEQ ID NO: 68 - DNA, 24 bp, Artificial sequence
  PCR primer
cagcaagttaagcaaaattcacca

SEQ ID NO: 69 - DNA, 22 bp, Artificial sequence
  PCR primer
gccgtgatcctccgagaagggg

SEQ ID NO: 70 - DNA, 24 bp, Artificial sequence
  PCR primer
tgatgatgctacagaaaggtgggg

SEQ ID NO: 71 - DNA, 27 bp, Artificial sequence
  PCR primer
ggctgggaagtttggtgatatagtgtc

SEQ ID NO: 72 - DNA, 21 bp, Artificial sequence
  PCR primer
atgatgaagtgtccacaggct

SEQ ID NO: 73 - DNA, 24 bp, Artificial sequence
  PCR primer
agcagccaaatccttcagtctcaa

SEQ ID NO: 74 - DNA, 28 bp, Artificial sequence
  PCR primer
aagacaaaaactcccccattgtgaaata

SEQ ID NO: 75 - DNA, 20 bp, Artificial sequence
  PCR primer
cagagttccccggaccagac

(19) United States
(12) Patent Application Publication
(10) Pub. No.: US 2008/0166704 A1
(43) Pub. Date: Jul. 10, 2008

(54) Method for Quantitative Evaluation of a Rearrangement or a Targeted Genetic Recombination of an Individual and Uses Thereof

(76) Inventors: Patrice Marche, Meylan (FR); Evelyne Jouvin-Marche, Meylan (FR); Nicolas Pasqual, Grenoble (FR)

Correspondence Address: MORGAN LEWIS & BOCKIUS LLP, 1111 PENNSYLVANIA AVENUE NW, WASHINGTON, DC, 20004, US

(21) Appl. No.: 10/581,814

(22) PCT Filed: Dec. 3, 2004
(86) PCT No.: PCT/FR04/03115

§ 371 Date: Aug. 22, 2007

(30) Foreign Application Priority Data
Dec. 5, 2003 (FR) 0314289

(51) Int. Cl. C12Q 1/68; C12Q 1/00; C07H 21/04

(52) U.S. Cl. 435/6; 435/4; 536/24.33; 536/24.3

(57) ABSTRACT

A method for quantitative evaluation of a rearrangement or targeted genetic recombination of an individual and the immunity repertoire of an individual and the uses thereof, particularly in the case of follow-up to a treatment or in the diagnosis and/or prognosis of certain pathologies. Said method comprises at least: (a) extraction of human genomic DNA from a biological sample, (b) amplification of a segment of said genomic DNA, the size thereof ranging from a few hundreds of base pairs and several quantities of ten kb by multiplex PCR in the presence of: one or several couples of primers, and polymerase DNA or a polymerase DNA mixture enabling the amplification of segments of genomic DNA, the size thereof ranging from several hundreds of base pairs and several quantities of ten kb, having a corrective effect enabling elongation to be substantially improved, said amplification also comprising a stage of initial denaturation, cycles of denaturation, hybridation and elongation and wherein the elongation stages are carried out at least during a period of 10 minutes at 68° C.-72° C.; (c) separation of the amplified DNAg fragments and (d) detection of the rearranged or recombined segments.

The present invention relates to a method for the quantitative evaluation of a rearrangement or of a targeted genetic recombination of an individual, and in particular of the immune repertoire of an individual, and to uses thereof, in particular in the case of follow-up to a treatment or in the diagnosis and/or prognosis of certain pathologies.

T and B lymphocytes play a predominant role in immune response adaptation. They are capable of recognizing a large number of antigens, by means of two groups of specific receptors: T-cell receptors (TCRs) and immunoglobulines (Igs). The TCRs are stimulated by the antigens, in the form of peptides bound to MHC class I or class II molecules and presented to said TCRs by the antigen-presenting cells (1, 2).

Each TCR (specific for an antigen) is expressed at the surface of T cells and consists of two heterodimers bound to the membrane and associated with the CD3 complex; these heterodimers are composed of two αβ or γδ polypeptide chains (3), attached to one another via a disulfide bridge; each chain comprises a variable domain (V) and a constant domain (C). The α and β or γ and δ domains of the variable regions form the antigen-binding zone (4, 5). The antigen recognition domains of the TCRs are generated in the T lymphocytes during their differentiation, essentially subsequent to site-specific somatic DNA recombination reactions, called V(D)J recombinations (9, 10). During T cell development, the α, β, γ and δ chains of the TCRs are assembled following rearrangement of their appropriate genes, independently of the various loci (TCRAD, TCRB, TCRG). In humans, the TCR genes are located on chromosomes 7 and 14: the genes encoding the TCRD chain are located in the locus of the TCRA chain on chromosome 14q11-12, whereas the genes encoding the TCRB and TCRG chains are located on chromosome 7, at positions 7q32-35 and 7p15, respectively. The four loci encode the constant and variable domains of the four chains. The V domains in the TCRA chain are assembled from V and J gene segments.

Thus, all the T lymphocytes define an immune repertoire which constitutes the register of the various forms of the receptors and of their antigenic specificity. The repertoire is based on the great diversity of the antigen receptor structures. To generate this diversity, lymphocytes have several mechanisms, the main of which is V(D)J recombination. Briefly, the genes encoding the antigen receptors are discontinued at the level of the genome of the organism's cells such that these genes are inactive. Several gene segments are distinguished:

a) the constant region (C), which is common to all the receptors of a family, regardless of their specificity,
b) the variable region (V), the number of which ranges depending on the TCR chain under consideration, from a few genes to several hundred genes,
c) the junction region (J), which is an intermediate gene between V and C, the number of which ranges from 1 to several tens, depending on the TCR chain under consideration and, finally,
d) the diversity region (D), which is a small gene of a few nucleotides present only in the β and δ chains of the TCRs and which intercalates between V and J.

The rearrangement process uses an enzymatic complex (V(D)J recombinase) (14), which specifically targets the recombination signal sequences (RSSs) flanking the sequences encoding the dispersed V and J gene segments.

More specifically, the main components of the recombinase are the products of two recombination activating genes RAG-1 and RAG-2 (15, 16). The RAG-1 and RAG-2 proteins bind to conserved nucleotide motifs, called recombination signal sequences (RSSs), which flank each V, D and J gene (17, 18).

The consensus RSS consists of a heptameric sequence directly adjacent to the coding element and of a nonameric sequence separated by a spacer arm comprising 12 or 23 relatively nonconserved nucleotides. The junction sites are determined by the RSSs and the established preference for recombination between an RSS with a spacer arm of 12 nucleotides and an RSS with a spacer arm of 23 nucleotides ensures a V(D)J recombination between two segments of different types (Vα and Jα, for example) (19, 20).

More generally, V(D)J recombination comprises a set of enzymatic reactions including specific cleavages, exonuclease and polymerase activities and DNA ligations, which lead to the formation of one functional gene entity per lymphocyte. This functional gene consists of the C region and of a combination of a V and of a J (and optionally D), which constitutes the molecular identity mark of the lymphocyte and the molecular basis of the specificity of the antigen receptor.

Lymphocytes thus have a sophisticated mechanism for generating a diversified TCR repertoire.

In addition to the recombination process (combinatory diversity), deletions or insertions are also observed at the V-J or V-DJ junctions (9, 21). These various mechanisms make it possible to increase the diversity of the TCR repertoire and therefore the number of antigens recognized. The latter mechanism is not encoded, but considerably increases the TCR repertoire.

Another diversification factor is the coupling of the a and β (or γ and δ) chains (22) so as to form the TCR heterodimer.

Most T lymphocytes express a clonotypic TCRαβ which is composed of an αβ heterodimer bound to the membrane.

Each chain contains a constant domain and a variable domain, the latter being responsible for the MHC-peptide recognition, which takes place in a manner that is always similar, by interaction with the CDR (complementarity-determining region) loops of the V domains.

Several geometries can be observed, depending on the composition of the V domains and the MHC-peptide complex recognized. For example, it has been shown that the TCR is positioned in a diagonal orientation above the face of the peptide-MHC complex, with the CDR1 and CDR2 loops of the TCRα chain positioned above the N-terminal half of the peptide and the corresponding regions of the TCRβ being positioned at the level of the C-terminal half of said peptide. A certain number of the interactions of CDR1 and CDR2 take place with the residues of the MHC.

The most variable loops of the TCR, i.e. the CDR3 loops of the TCRα and TCRβ chains, are positioned centrally and are preferably in contact with the peptide.

Thus, the potential diversity of TCRs-αβ generated by V(D)J random recombination is estimated to be approximately 10¹⁵ (2, 23) if any V gene rearranges with any D and/or J gene (2, 23).

The theoretical diversity has been overestimated at several levels:
the evaluation of the T repertoire was calculated with the hypothesis that any V gene can rearrange with any D and/or J gene.

However, recent data obtained from mouse thymus has shown that the number of V-Jα recombinations is considerably less than that predicted by the random rearrangement model and that preferential associations exist between the Vα and Jα segments according to their location on the locus, resulting in a regulated and coordinated use (24);
a restriction exists in the preparing of the α and β chains so as to form heterodimer receptors, due to structural compatibilities between the various subunits, which also limits the repertoire;
in the thymus, the repertoire generated is subjected both to a positive selection and to a negative selection due to the existence of interactions with the MHC molecules expressed on the stromal cells. These selections also reduce the size of the repertoire generated, approximately by a factor of 100, and provide the profile of diversity of mature T cells. These lymphocytes (CD4⁺ or CD8⁺) migrate to the periphery, where they constitute the naïve pool of circulating T lymphocytes.

The peripheral T lymphocyte pool is subjected to complex homeostatic mechanisms which make it possible to maintain the number and the function of the lymphocyte populations.

This includes the control of production rate, mature cell division, intracellular trafficking and cell death.

The establishment of a peripheral T repertoire in normal individuals is thus not only based on the interactions of each T cell with its respective ligands, but also on competition with the other lymphocyte subpopulations.

Thus, the size of peripheral TCRαβ diversity is difficult to determine.

Estimations have recently been obtained by extrapolation from molecular measurements of TCR diversity using the analysis of CDR3 segment length.

However, these analyses concern mainly the diversity of the β chain due to the complexity of the locus of the Vα chain.

The human TCRAD locus comprises approximately 1000 kb and consists of 54 Vα genes belonging to 41 families and 8 pseudogenes, 61 Jα genes including 3 J pseudogenes, and a single Cα gene (25, 26).

Despite a high degree of sequence identity between the human and mouse TCR loci, the organization of the V and J genes is only partially conserved between the two species.

Although the number of J gene segments for the α and β chains is well conserved in the two species, significant differences exist in the number of Vα and Vβ segments.

In particular, there are at least twice as many Vα genes in the mouse than in humans and ⅓ more Vβ genes in humans compared with the mouse.

During evolution, the mouse TCRAD locus was subjected to multiple duplication processes involving large portions of the locus, whereas, in humans, the duplication occurred only in limited parts of the TCRAD locus (26).

These various processes show that the data observed in the mouse are not directly transposable to humans.

Analysis of the immune repertoire in humans has been proposed, for diagnostic purposes.

For example, Hodges E. et al. (46) summarize the diagnostic role of T-cell receptor (TCR) gene evaluation tests. In particular, the authors of this article show that it is now possible to study the pathogenesis of a disease at the genomic level. T-cell receptor (TCR) gene rearrangement is an important event in T cell ontogeny, which allows T cells to recognize antigens specifically.

The slightest deregulation in this highly complex process can result in a disease.

This article reviews the knowledge concerning the TCR gene rearrangement mechanism and describes disorders in which clonal expansions and proliferations of T cells are observed.

The methods currently used for studying the various T cell populations, their diagnostic role and the limitations of these methods are also disclosed in this article.

Table I of this document summarizes the various genes encoding the TCR chains: α(A), β(B), γ(G) and δ(D), and shows in particular the complexity of the TCRAD locus. In normal individuals, the TCR repertoire is stable and polyclonal, whereas clonal populations are the sign of a specific immune response against a tumor pathology or an infectious pathology. Thus, clonal and oligoclonal populations are observed under non-tumor conditions, such as HIV or EBV infections, and specific states, such as elderly individuals, autoimmunity, common variable immunodeficiency (CVID) and severe combined immunodeficiency (SCID), in which anomalies in the mechanisms involved in V(D)J recombination allow only a limited number of correct rearrangements of the TCR gene. The authors of this article consider that:
for studying the phenotype, flow cytometry constitutes a rapid, relatively inexpensive method for analyzing the TCR repertoire; furthermore, such a method is reproducible and allows a quantitative evaluation of the expression of various V genes in the various T cell subsets (CD4+ and CD8+ cells).
for studying the genotype, PCR, which has largely replaced Southern blotting analysis, is useful for clonality studies, insofar as PCR offers a certain number of advantages, and in particular rapid implementation and the possibility of using a small amount of DNA.

Several different PCR protocols can advantageously be used:

1. Amplification of all the V segments using primers specific for families complementary to all the TCRV genes and a primer located in the constant region (semi-quantitative method only) (French patent application No. 2 671 356; Panzara M. A. et al., Biotechniques, 1992, 12, 5, 728-735 and Langerak A. W. et al., Blood, 2001, 98, 165-173).

2. A PCR which includes a step consisting in modification of all the TCR transcripts, such that they can be amplified by a single oligonucleotide (anchored PCR).

3. A PCR that allows the production of circular transcripts by ligation.

The last two methods require, in order to be carried out, a high level of technical expertise (problem of routine use), but allow a quantitative analysis of the T cell population, and have already been applied to studies of clonality and to evaluation of the TCR repertoire in various pathologies.

It emerges from this article that the methods for evaluating clonality, routinely, must preferably satisfy the following characteristics:
simplicity;
robustness with good detection yields and good sensitivity;
implementation of the test on DNA;
increase in the detection and in the sensitivity by using selected primers.

It also emerges from this article:
that PCR analysis of the TCRG genes satisfies these conditions (studies of clonality in clinical pathology) because of the limited repertoire of the TCRG genes, which thus decreases the number of PCR primers required. However, this choice has the drawback of bringing about parasitic amplifications (similar rearrangements in normal cells);
that PCR analysis of the TCRD genes is a useful tool in the analysis of certain tumors, and in particular of MRDs;
that the TCRB locus is the locus of choice for establishing the clonality of tumors expressing TCRαβ, whereas
the TCRA locus is too complex for clonal DNA analyses.

This is the reason for which the various methods for analyzing TCR repertoires currently proposed never recommend analysis of the TCRA repertoire:
Analysis by Southern transfer (DNA)

This technique is laborious to implement, not very thorough and not very quantitative. It makes it possible to evaluate V(D)J rearrangements, but it is not very sensitive since it requires several μg of DNA and, furthermore, it provides little resolution for identifying V and J genes. Finally, it lends itself poorly to automation.
Analysis by Immunoscope® (patent U.S. Pat. No. 5,635,354; Pannetier et al., Immunol. Today, 1995, 16, 4, 176-181); this method is uniquely qualitative.
Analysis of the diversity of “CDR3” recombination regions by Immunoscope® (mRNA) (PCT international application WO 02/084567).

Currently, this technique is the most popular for evaluating the diversity of the antigen receptors. It is based on the use of RNAs which, once converted to cDNA by reverse transcriptase, are subjected to PCR amplification reactions. The amplified gene fragments are short (a few tens of nucleotides), and they extend between the V gene and the C or J gene and include CDR3, which is the most polymorphic region of the antigen receptors. It makes it possible to estimate the diversity generated during the rearrangements. It gives an indication of the relative diversity of a repertoire, without being able to quantify the entire repertoire. The weak points of this technique lie in:

1) the use of RNA, which is a genetic material that is readily degraded and requires specific handling conditions,
2) the fact that bias can exist in the evaluation of the rearrangements due to the fact that not all the V genes are necessarily transcribed with the same efficiency,
3) the synthesis of cDNA can vary according to transcripts; this step can therefore introduce a bias that is difficult to control.

This technique requires quite a large number of amplification reactions making it difficult to completely analyze the diversity of a receptor. Thus, for the beta chain of the human TCR, 25 independent reactions would be necessary for the Vs, and each one of them would then have to be repeated with one of the 13 functional Js, for an electrophoresis analysis on denaturing polyacrylamide gel.
Analysis of the V-J junction

PCT international application WO 2004/033728, which benefits from a filing date of Oct. 11, 2002, but was published on Apr. 22, 2004, i.e. after the filing date from which the present application benefits, describes a method that proposes standard multiplex PCR primer combinations and also standardized protocols for detecting clonal recombinations of Ig genes and T-cell receptor (TCR) genes in samples where a proliferation of the lymphoid line is suspected. The method described uses the multiplex PCR technique under PCR conditions that generate a product, the size of which is between 300 and 700 pb, and is preferably less than 300 pb (more preferably between 100 and 300 pb); more specifically, the PCR used comprises an elongation step of 1 min 30 which allows an amplification of a maximum of 1 to 2 Kb and makes it impossible to amplify the large sizes necessary for “natural” multiplexing; in addition, the position of the oligonucleotides used targets regions that are conserved between V genes; the various genes which have been tested in this method are as follows: TCRB, TCRG, TCRD. They do not include the TCRA gene, which was intentionally discarded because of its complexity.

This method makes it possible to characterize monoclonal amplifications; on the other hand, it does not make it possible to obtain a resolution of the entire repertoire. This approach therefore gives an on/off-type result, which gives little resolution and characterizes a monoclonal amplification, but in no way makes it possible to visualize the entire repertoire in the form of a mapping.

This technique does not therefore make it possible to determine the relative frequency of the rearrangements of the repertoire due to the fact that only the V-S junction is studied, and does not provide any differentiating aspect with respect to a given rearrangement. In fact, the fact of having multiplexed all the oligonucleotides in the same tube, without preventing the superimposition of the various PCR products, only makes it possible to obtain a product from 200 to 700 pb in size for all the rearrangements expected, and prevents the assignment of a band to a specific rearrangement. This technique in no way uses the “natural” multiplexing principle of TCR loci.
Flow cytometry (see Hodges E. et al., mentioned above, 46)

This technique uses fluorescence-labeled antibodies specific for receptor V regions. It has the advantage of measuring the expression of the antigen receptor at the surface of the lymphocyte. However, it is very limited:
by the lack of antibodies, which does not allow a complete analysis of the repertoire;
by its weak sensitivity, which requires large cell samples (several tens of millions of lymphocytes);
by its poor resolution, since it only makes it possible to identify the V without giving any information on the combinations with Js;
by the need to work with cells.

Consequently, the present invention gave itself the aim of providing a method for the evaluation of rearrangements or recombinations of genes, and in particular the TCR receptor, which correspond better to the practical needs than the abovementioned methods of the prior art.

To obtain a quantitative method which is simple to carry out, robust, reliable and quantitative, the present invention recommends using, under defined conditions, a restricted number of enzymatic DNA polymerization chain reactions, referred to as “long PCR” (LPCR), for detecting several gene rearrangements, and in particular several V(D)J rearrangements of the genes encoding TCRADs directly at the level of the genomic DNA with a single enzymatic reaction (multiplex PCR).

The invention thus uses the amplification of gDNA for detecting, in a single step, the rearrangements of V(D)J genes encoding antigen-specific receptors. The invention can be applied to other gene rearrangement or genetic recombination events having defined sites.

Consequently, the invention also relates to a method for the evaluation of any targeted genetic recombination.

A subject of the present invention is therefore a method for the quantitative evaluation of a rearrangement or of a targeted genetic recombination of an individual, which method is characterized in that it comprises at least:

(a) the extraction of human genomic DNA from a biological sample,
(b) the amplification of a segment of said genomic DNA, between a few hundred base pairs and several tens of kb in size, by multiplex PCR, in the presence:
of one or more pairs of primers, selected so as to correspond to the following characteristics:
at least one of said primers of one of said pairs of primers hybridizes upstream and/or at the 5′ end of a Vx gene to be amplified, which may be involved in said genetic rearrangement;
at least the other of said primers of one of said pairs of primers hybridizes downstream and/or at the 3′ end of a Jy gene to be amplified, which may be involved in said genetic rearrangement;
and of a DNA polymerase or a mixture of DNA polymerases for amplifying genomic DNA segments between a few hundred base pairs and several tens of kb in size, preferably greater than 10 kb in size, and having a correction activity that makes it possible to substantially improve the elongation;
   said amplification comprising, in addition to the initial denaturation step, cycles of denaturation, hybridization and elongation, in which the elongation steps are carried out at least for 10 minutes at 68° C.-72° C.;
   c) the separation of the gDNA fragments amplified, and
   d) the detection of the rearranged or recombined segments.

The term “individual” is intended to mean preferably mammals for which there is an advantage in determining TCRA expression profiles (humans, other primates, domestic animals, animals used in the food chain, such as cattle, sheep, pigs, etc.).

When the rearrangement or the targeted genetic recombination concerns TCRAD receptors, said method comprises:

a) the extraction of human genomic DNA from a biological sample,
b) the amplification of a segment of said genomic DNA, between a few hundred base pairs and several tens of kb in size, by multiplex long PCR, in the presence:
of one or more pairs of primers, selected so as to correspond to the following characteristics:
at least one of said primers of one of said pairs of primers, called primer V, hybridizes specifically with a region located upstream of the RSS sequence of a Vx gene to be amplified, corresponding to a V segment of the variable domain of the α chain of a T-cell receptor (TCRAD);
at least one of said primers of one of said pairs of primers, called primer J, hybridizes specifically with a region located downstream of the RSS sequence of a Jy gene to be amplified, with the 3′ end of said Jy gene to be amplified or in said Jy gene to be amplified, corresponding to a J segment of the α chain of a T-cell receptor;
and of a DNA polymerase or a mixture of DNA polymerases for amplifying genomic DNA segments between a few hundred base pairs and several tens of kb in size, preferably greater than 10 kb in size, and having a correction activity that makes it possible to substantially improve the elongation;
   said amplification comprising, in addition to the initial denaturation step, cycles of denaturation, hybridization and elongation, in which the elongation steps are carried out at least for 10 minutes at 68° C.-72° C.;
   (c) the separation of the gDNA fragments amplified, and
   (d) the detection of the recombined V(D)J segments.

This method has a certain number of advantages:
it allows an analysis of the V(D)J combinations at the level of the genome, something which the Immunoscope® technique does not;
it makes it possible to obtain large fragments, it being possible for each Vₓ-J_{y}, Vₓ-J_{y+1}, Vₓ-J_{y+2} etc. fragment to be clearly distinguished from the others, which implies good resolution;
it thus allows the analysis of V/J pairing as such;
it allows a molecular analysis at the genomic DNA level. By measuring the amount of a given combination of V(D)J genes, it is possible to obtain an evaluation of the number of lymphocytes having this rearrangement, since there is a proportionality between the number of rearranged genes and the number of lymphocytes: a lymphocyte can have only two rearrangements for a type of receptor, i.e. one rearrangement per chromosome. This type of information is not accessible through analysis of the diversity of the “CDR3” recombination regions, or through analysis of the V/J junction;
it allows a thorough evaluation of the diversity of antigen receptors, which neither flow cytometry nor the Southern technique allow;
it makes it possible to substantially decrease the number of PCR reactions for complete analysis of the repertoire. This is possible by virtue of the simultaneous analysis of several V and J genes in the same amplification reaction and the same electrophoresis lane. For example, for the TCR alpha chain in humans, 3355 reactions corresponding to the various VJ combinations are required in order to detect and quantify the rearrangements; the method according to the invention makes it possible to carry out the analysis with ten times fewer reactions. In particular, the combination of the steps of the method according to the invention, and more particularly the combination of the separation step (c) with the amplification step (b), cooperates so as to significantly improve the effectiveness of the method according to the invention, in particular by selecting the most suitable separation system;
it makes it possible to quantify the rearrangements by measuring the relative intensity of the electrophoresis bands derived from the same reaction and which are analyzed in the same electrophoresis lane. By virtue of a standardization taking into account the differences in size, it is possible in particular to normalize the values and to obtain the quantification of the proportion of each rearrangement relative to the whole (very good resolution). No technique currently makes it possible to obtain these results;
it allows the identification of the V and J genes of the receptor, the identity of the V gene is given by the primer used during the amplification, the identity of the J gene is obtained by measuring the size of the fragment amplified with the primer J used and by reference to the published nucleotide sequence. This operation is facilitated by the use of sequence alignment and motif comparison software such as Blast (www.ensembl.org), IMGT/GeneInfo (http://imgt.cines.fr/GeneInfo), etc. This information can be obtained by analysis with Immunoscope®, but the identification of the J segment is carried out case by case, whereas, with the method according to the invention, several J segments can be identified in the same reaction;
it makes it possible to reveal V(D)J combinations that are particularly abundant in a mixture. This can prove to be useful for detecting an immunodominant specific response, i.e. a response due to one (monoclonal response) or even to several (oligoclonal response) lymphocyte clones. In addition, this can be used for the detection of lymphocyte proliferations such as those observed in lymphocyte cancers (lymphomas, myelomas, leukemias). Leukemias originating from B lymphomas can be detected by direct analysis of circulating Igs in the blood serum. However, the detection of leukemias that do not produce Ig in the serum and that of T lymphoma leukemias remain difficult and not very quantitative. The method according to the invention makes it possible to identify V(D)J combinations that are abnormally abundant and thus allows the detection of proliferations due to leukemias. Through analysis at the level of the genome, the method according to the invention allows an easier and more sensitive detection of the number of cancer cells derived from B and/or T lymphocytes, and, consequently, makes it possible to provide an earlier and more thorough diagnosis, compared with the techniques already existing such as the Southern or Immunoscope® technique, of the TCR beta chain.

The extraction step (a) is carried out by known techniques which observe the precautions designed for the purification of gDNA, used for the Southern transfer technique (see Sambrook J., Fritsch E. F., Maniatis T., 1989, Molecular cloning. A laboratory manual. Second Edition. Cold Spring Harbor, N.Y., USA). It can be advantageously followed by a step consisting of purification of the gDNA by conventional molecular biology techniques.

According to an advantageous embodiment of the amplification step (b), the selection of the primers is carried out:
by systematic analysis of the entire locus concerned, and in particular of the human TCRAD locus, using a suitable sequence alignment and motif comparison software, as defined above, and in particular using a homologous sequence search software (“Vector NTI suite 8.0” from the company Informax Inc., for example),
selection of the primers whose 3′OH end is complementary only to the region of interest, as defined above,
elimination of the primers forming autodimers or stable hairpins, in particular by analysis using a suitable sequence alignment and motif comparison software, and in particular using a homologous sequence search software (the “vector NTI suite 8.0” software from the company Informax Inc., for example), and
elimination of the pairs of primers which form hybrids with one another.

Thus, all these various selection steps decrease the biases of the PCR, such as competition between the primers or with other target sequences other than those selected, and, consequently, improve the yield and the specificity of the PCR required for carrying out the rearrangement analyses in multiplex long PCR.

In accordance with the invention, the primers, and in particular the primers V and J of the pairs of primers V/J, are selected from the group consisting of the primers illustrated in table I below:

| | | | | |
|---|---|---|---|---|
| species | HUMAN | | | |
| Locus | TCRAD | S/A | bp | tm |

| | | | | | |
|---|---|---|---|---|---|
| TRAV | | | | | |
| hTRAV1 | GGTCGTTTTTCTTCATTCCTTAGTCG | (SEQ ID NO: 1) | S | 27 | 58 |
| hTRAV2 | TCTCTTCATCGCTGCTCATCCTCC | (SEQ ID NO: 2) | S | 24 | 61 |
| hTRAV3 | TCCCCTTCCCATTTTCCACTCG | (SEQ ID NO: 3) | S | 22 | 60 |
| hTRAV5 | GCACTTACACAGACAGCTCCTCCACC | (SEQ ID NO: 4) | S | 26 | 61 |
| hTRAV8 | CAGGAGGAACCAGAGCCCAGTC | (SEQ ID NO: 5) | S | 22 | 59 |
| hTRAV26-2 | TGGAGTAGGGCAGGGAGGACAGT | (SEQ ID NO: 6) | S | 23 | 60 |
| hTRAV35 | Ggctgggaagtttggtgatatagtgtc | (SEQ ID NO: 7) | S | 27 | 59 |
| hTRAV38.2 | AGCAGCCAAATCCTTCAGTCTCAA | (SEQ ID NO: 8) | S | 24 | 58 |
| hTRAV40 | Aagacaaaaactcccccattgtgaaata | (SEQ ID NO: 9) | S | 26 | 60 |
| hTRAV41 | GCCCTCCTGAAAATGTGTAAAGAAATGT | (SEQ ID NO: 10) | S | 28 | 60 |
| TRAJ_do | | | | | |
| hAJ53do | CTTCCCCCACTCCCTTCAAACTTAC | (SEQ ID NO: 11) | A | 25 | 60 |
| hAJ48do | AGCACTTGACGGCAGCAGCA | (SEQ ID NO: 12) | A | 20 | 60 |
| hAJ41do | TGCCCCGAGACCTGATAACCAA | (SEQ ID NO: 13) | A | 22 | 61 |
| hAJ29do | TCAGAACAAGCTGGAGGCAACTAGG | (SEQ ID NO: 14) | A | 25 | 61 |
| hAJ18do | GGAATAGAAAGCGACTCACTCACCAGG | (SEQ ID NO: 15) | A | 27 | 60 |
| hAJ10do | CCACTTTTAGCTGAGTGCCTGTCCC | (SEQ ID NO: 16) | A | 25 | 60 |
| hAJ5do | CTGTCTCTGCAATGATGAAATGGCC | (SEQ ID NO: 17) | A | 25 | 59 |
| hAJ1do | GGAAACTCTGGGCATGGGCAG | (SEQ ID NO: 18) | A | 21 | 59 |
| hAJ56do | ACTGGGCAGGAGATTCGGTTAT | (SEQ ID NO: 19) | | | |
| hAJ33do | CGCCCCAGATTAACTGATAGTTGCT | (SEQ ID NO: 20) | | | |
| hAJ24do | ATACTAAGGGCAGGTGAGGCTCCA | (SEQ ID NO: 21) | | | |

Thus, the primers selected in step (b) make it possible to obtain quality results: the specificity of the PCR products is more particularly established for 10 primers V (SEQ ID NO: 1-10), by sequencing of the amplification products, which shows the presence of a unique sequence corresponding to the targeted V gene (see table III) and the robustness of the choice of primers.

The specificity of the primers can be monitored using labeled oligonucleotide probes, such as those defined in table II below, which recognize sequences internal to the product amplified; by hybridization, it is thus possible to verify whether the observed size of the amplified fragment corresponds to the expected size deduced from the sequence of the human TCR alpha locus, taking into consideration the distance separating the primers V and J and the rearrangement thereof. These probes are also advantageously used in step (d) of the method according to the invention.

TABLE II

| | | | |
|---|---|---|---|
| Probes | S/A | bp | tm |

| | | | | | |
|---|---|---|---|---|---|
| TRAV | | | | | |
| hTRAV1p | TCGTTTTTCTTCATTCCTTAGTCG | (SEQ ID NO: 22) | | | |
| hTRAV5p | ATGAAACAAGACCAAAGACTCACTG | (SEQ ID NO: 23) | | | |
| hTRAV8p | TGACCCAGCTTGAGAGCCA | (SEQ ID NO: 24) | | | |
| hTRAV26-2p | GGCAATCGCTGAAGACAGAAAG | (SEQ ID NO: 25) | | | |
| hTRAV41p | GAGAACAGGTGTAAGTGCCGCC | (SEQ ID NO: 26) | | | |
| TRAJ | | | | | |
| hAJ53p | TTGGATTCACGGTTAAGAGAGTTC | (SEQ ID NO: 27) | A | 24 | 59 |
| hAJ48p | TCCAGTCCCAAAGGTTAATTTCTC | (SEQ ID NO: 28) | A | 24 | 59 |
| hAJ41p | CCGAAGTTGAGTGCATACCCG | (SEQ ID NO: 29) | A | 21 | 55 |
| hAJ29p | CAAAATCAAGGATGGCTAGAAACAC | (SEQ ID NO: 30) | A | 25 | 55 |
| hAJ18p | CTTCCAAAGTATAGCCTCCCCAG | (SEQ ID NO: 31) | A | 23 | 55 |
| hAJ10p | GGTGAGTTTGTTTCCTCCTCCC | (SEQ ID NO: 32) | A | 22 | 57 |
| hAJ5p | CCCAAAAGTAAGTGCTCTCCTGCC | (SEQ ID NO: 33) | A | 24 | 55 |
| hAJ1p | CGGGGAGAAGTGGAAACTCTGG | (SEQ ID NO: 34) | A | 22 | 53 |
| hAJ56p | TCAGAGTTATTCCTTTTCCAAATG | (SEQ ID NO: 35) | | | |
| hAJ33p | CGCCCCAGATTAACTGATAGTTGCT | (SEQ ID NO: 36) | | | |
| hAJ24p | GGTCCCTGCTCCAAACTGC | (SEQ ID NO: 37) | | | |

The probes and primers according to the invention can be labeled directly or indirectly with a radioactive or nonradioactive compound, by methods well known to those skilled in the art, in order to obtain a detectable and/or quantifiable signal; the labeling of the primers or of the probes according to the invention is carried out with radioactive elements or with nonradioactive molecules. Among the radioactive isotopes used, mention may be made of ³²P, ³³P, ³⁵S or ³H. The nonradioactive entities are selected from ligands such as biotin, avidin, streptavidin or digoxigenin, haptenes, dyes, and luminescent agents such as radioluminescent, chemoluminescent, bioluminescent, fluorescent or phosphorescent agents.

To prevent amplification of neighboring sequences outside the TCRAD locus, and therefore to decrease the nonspecificity, the systematic analysis carried out over the entire human TCRAD locus in order to select the primers, in step (b), is preferably carried out by means of a search over the entire human genome using the www.ensembl.org database, which makes it possible to visualize all fixes on the human genome with a 3-intensity color code: red=100%, green=85, blue=70.

Only the candidate oligonucleotides that recognize the TCRAD locus in red and have a limited number of fixes on the rest of the genome (in green or blue) are selected.

The products from amplification by PCR using the primers specified in table III below provide the amplified sequences specified in the right-hand column.

TABLE III

| | | Amplified V sequences: |
|---|---|---|
| oligo up | oligo do | SEQ ID NO: 38-45 |
| SEQ ID NO: 6 | SEQ ID NO: 11 | CTTGAGAGATGCTGCTGTGTACT |
| | | ACTGCATCCTGAGAGACGGGGGG |
| | | GGGG |
| SEQ ID NO: 1 | SEQ ID NO: 16 | CCTTTTGAGGAGCTCCAGATGAA |
| | | AGACTCTGCCTCTTACCTCTGTG |
| | | CTGTGAGGAATGGGGGGG |
| SEQ ID NO: 8 | SEQ ID NO: 11 | GCGATGTATTTCTGTGCTTACAT |
| | | GAGCCCGGGGGGGGGGGG |
| SEQ ID NO: 10 | SEQ ID NO: 11 | TATTCTGTATCTGATGATGTCTT |
| | | TGAGAACAGGTGTAAGTGCCGCC |
| | | AAAAATGAGTGGAGCAGAGTCCT |
| | | CAGCCTGACTGCCCAGGAAGGAG |
| | | AATTTATCACAATCAACTGCAGT |
| | | TACTCGGTAGGAATAAGTGCCTT |
| | | ACACTGGCTGCAACAGCATCCAG |
| | | GAGGAGGCATTGTTTCCTTGTTT |
| | | ATGCTGAGCTCAGGGAAGAAGAA |
| | | GCATGGAAGATTAATTGCCACAA |
| | | TAAACATACAGGAAAAGCACAGC |
| | | TCCCTGCACATCACAGCCTCCCA |
| | | TCCCAGAGACTCTGCCGTCTACA |
| | | TCTGTGCTGTCAGAGGGGGGGGG |
| | | GGG |
| SEQ ID NO: 9 | SEQ ID NO: 11 | ACTCAGCCGTGTACTACTGTCTT |
| | | CTGGGAGATGGGGGGGGGGGG |
| SEQ ID NO: 4 | SEQ ID NO: 16 | ACCTGGAGCAGGTCTCCAGCTTG |
| | | CTGACGTATATTTTTTCAAATAT |
| | | GGACATGAAACAAGACCAAAGAC |
| | | TCACTGTTCTATTGAATAAAAAG |
| | | GATAACATCTGTCTCTGCGCATT |
| | | GCAGACACCCAGACTGGGGACTC |
| | | AGCTATCTACTTCTGTGCAGAGA |
| | | G |
| SEQ ID NO: 5 | SEQ ID NO: 11 | ACTACTCATCGTCTGTTTCACNG |
| | | TATCTCTTCTGGTATGTGCAATA |
| | | CCCCAACCAAGGACTCCAGCTTC |
| | | TCCTGAAGTANNNATCAGGNNCC |
| | | ACCCTGGTTAAAGGCATCAACGG |
| | | TTTTGAGGCTGAATTTAACAAGA |
| | | GTGAAACNTCCTTCCACNTGANG |
| | | AAACCCTCAGCCCATATNAGCGA |
| | | CNCGGCTGAGTACTTCTGTGCTG |
| | | TGAGTGAT |
| SEQ ID NO: 4 | SEQ ID NO: 11 | ACCTGGAGCAGGTCTCCAGTTGC |
| | | TGACGTATATTTTTTCAAATATG |
| | | GACATGAACAAGACCAAAGACTC |
| | | ACTGTTCTATTGAATAAAAAGGA |
| | | TAAACATCTGTCTCTGCGCATTG |
| | | CAGACACCCAGACTGGGGACTCA |
| | | GCTATCTACTTCTGTGCAGAGAG |
| | | T |

For each Vx gene, it is preferable to use the pairs of primers comprising the primer V corresponding to said Vx gene in combination with each of the primers J defined above (SEQ ID NO: 11 to 22). The amplification step (b) is then carried out in parallel with the various pairs of primers.

According to another embodiment of the method according to the invention, step (b) can advantageously use additional primers for amplifying, in addition, at least one of the following segments: D segments, V segments and J segments of the TCR β, γ, δ chains and, optionally, segments of the immunoglobulin chains.

Thus, in particular in the case of TCR chains or Ig chains comprising a D segment having diversity, a PCR reaction between the V and D gene is carried out in addition to the V-J reaction common to all the rearrangements. This step makes it possible to determine the repertoire of both the D and J segments used in conjunction with the V genes. The same is true for the D genes, in the case of the analysis of chains which have Ds in addition to the Vs and Js, like the TCR beta and TCR delta chains and the immunoglobulin heavy chain.

The DNA polymerases used in the amplification step (b) are preferably a mixture of DNA polymerases for amplifying very long DNA molecules, of the type such as that described in American patent U.S. Pat. No. 6,410,277; such a mixture of enzymes has a correction activity which makes it possible to substantially improve elongation. Thus, an amplification of several kb of DNA makes it possible to detect the successive recombinations having taken place with all the potential genes located between the two primers used.

According to another advantageous embodiment of step (b), the multiplex long PCR (LPCR) reaction is carried out after purification of the DNA by conventional molecular biology techniques, or directly on a cell lysate.

It is, however, preferable to work with gDNA that is as pure as possible (free of proteins such as nucleosomes) so as to promote optimum amplification of the large products (>10 kb); for this, it is important not to break the genomic DNA.

Once the purification of the DNA has been carried out (either by means of a method of extraction with phenol/chloroform V/V well known in molecular biology, or with commercial kits for extracting and purifying DNA), the latter is directly used for the multiplex LPCR.

A reaction tube (reaction volume of 20 to 50 μl) is composed of ultrapure water, the Taq enzyme or the mixture of DNA polymerization enzymes such as that described in patent U.S. Pat. No. 6,410,277 (mixture called LATaq), MgCl₂ (Taq enzyme cofactor), dNTPs, the oligonucleotide primer specific for the direction of transcription of the V genes to be studied (one of the primers of SEQ ID NO: 1-10), the oligonucleotide primer specific for the reverse direction of transcription of the D or J gene segment (one of the primers of SEQ ID NO: 11-21) to be studied, and the DNA to be analyzed, also called matrix, from which the amplification takes place. A second oligonucleotide corresponding to an internal sequence of the V gene, located downstream of the first primer V and upstream of the RSS of the V gene and/or a second nucleotide J corresponding to an internal sequence of the J gene, located upstream of the first primer J and downstream of the RSS of the J gene, can be added to half the LPCR reaction in order to decrease the PCR background noise; these oligonucleotides may or may not be labeled with a fluorochrome so as to allow direct detection of the amplified products. The program of the thermocycle for carrying out the amplification reaction can be variable according to the desired conditions.

One PCR (amplification step (b)) per pair of primers V/J, as defined above, is carried out.

According to another advantageous embodiment of the amplification step (b), the elongation steps are advantageously incremented by 15-20 seconds per additional elongation cycle.

However, in order to allow the amplification of long DNA fragments, it is necessary (step (b)):
to expose the genomic DNA to be amplified to temperatures above 90° C. (denaturation) for very short periods of time (of the order of 10 to 30 seconds), in order to prevent acidification of the medium and degradation of the DNA,
to take into account the “exhaustion” of the DNA polymerase used, by incrementing the elongation step by 15-20 seconds per additional cycle, in particular based on the PCR medium.

A nonlimiting example for amplifying products of the order of 10 kb to 20 kb is described below:
initial denaturation 1-2 minutes at 94° C.,
15 successive cycles comprising: a denaturation step: 5 to 30 seconds at 90-94° C., a primer hybridization step: 15-30 seconds at 58-62° C. and an elongation step: 14 to 20 minutes at 68-72° C., and
15 successive cycles comprising: a denaturation step: 5 to 30 seconds at 90-94° C., a primer hybridization step: 15-30 seconds at 58-62° C. and an elongation step: 14 to 20 minutes at 68-72° C.+15-20 additional seconds at each additional cycle, and
1 final elongation cycle of 10 minutes at 72° C.

According to another advantageous embodiment of said method, the step (c) consisting of separation of the amplified DNA fragments is carried out by electrophoretic migration on an agarose or poly-acrylamide gel, preferably pulsed-field migration.

The conditions for separating the PCR products can vary as a function of the conditions desired. The following is a condition for separating products of the order of 15 kb by migration on an agarose electrophoresis gel in 1×TBE (this step is well known in the state of the art):

Loading of 5-20 μl of LPCR product on a 1.2% (W/V) agarose gel, migration at 100-150 volts per 8 h with a power source. It is possible to use pulsed-field migration in order to improve the separation of the large products.

According to another advantageous embodiment of said method, the step (c) consisting of separation of the amplified DNA fragments is carried out by microcapillary separation on a bioanalyzer. More specifically, to separate LPCR fragments greater than 10 kb in size, microcapillary migration with an AGILENT Technologies bioanalyzer can be used. In such a case, the use of primers labeled with a fluorochrome (for example Cy-5) can be envisioned, in order to increase the signal of detection of the amplified fragments, in the detection step (d). The AGILENT chip is prepared and used according to the constructor's indications; deposition of 1 μl of the PCR product, quantification in accordance with the user's guide for the device.

Surprisingly, the combination of the following elements of step (b):
the choice of the sequences of the primers for studying the rearrangements of antigen receptors in humans;
the program of the thermocycle during the amplification reactions;
the choice of the enzymes used to improve the amplification of large fragments;
   and the protocol for separating the amplified fragments in step (c), effectively allow a quantitative evaluation of the recombinations to be detected.

In this context, it is possible to detect between 1 and 11 rearrangements per PCR reaction, and to visualize 100% of rearrangements effected in the J region, a minimum of 11 PCRs for a given V gene is sufficient.

According to another embodiment of said method, the detection step (d) can advantageously be carried out using one of the following methods:
by Southern transfer of the amplified products onto nylon membranes, followed by visualization after hybridization with one or more nucleotide probes specific for a sequence internal to the amplified product (probes of SEQ ID NO: 22-37, in particular) labeled with a radioactive isotope or a fluorochrome; more specifically, this method comprises Southern transfer onto a nylon or cellulose nitrate membrane by blotting paper capillary action, for example with a 20×SSC buffer, crosslinking of the gDNA onto the membrane after exposure to 700 kjoules of UV radiation (step well known in the state of the art), prehybridization with hybridization medium under conditions that make it possible to decrease the nonspecific background noise, hybridization with one or more radioactively labeled internal oligonucleotide probes (T4 kinase+³² PgammaATP method) for specifically revealing the PCR products, and validation of the specificity of the rearrangements by measuring the size of the PCR products. Relative quantification of the intensity of each DNA band corresponding to the various rearrangements using an imager and suitable software such as Quantity-One from the company Biorad;
by using a labeled base (labeled with a radioactive isotope or a fluorochrome) during the amplification and then by measuring the incorporation thereof directly in the gel;
by using a DNA-labeling agent (such as ethidium bromide, SybrGreen I, or the like) during the migration, and detecting after excitation in the UV range or at another appropriate wavelength;
by using oligonucleotides labeled with fluorochromes or other enzymatic revealing means (for example, avidin-biotin, peroxidase, etc.) during the amplification.

Any other method of detection can also be envisioned.

According to an advantageous arrangement of this embodiment, the probes are advantageously selected from the group consisting of the sequences SEQ ID NO: 22-37, as defined in table II.

A given rearrangement is defined from the amplification products: firstly, according to the distance of their migration and, secondly, by hybridization with specific internal probes. It is possible to individually detect several rearrangements in a single reaction. By combining a sufficient number of reactions, it is possible to quantify all the V(D)J rearrangements constituting the immune repertoire directly at the genomic DNA level.

Advantageously, the method according to the invention can be carried out on various types of biological samples: cells in culture, cell lines, samples originating from biopsies, from microdissection, from blood samples, from sorted cells; preferably, on blood samples.

The method according to the invention, as defined above, allows in particular the total or partial analysis of the immune repertoire by analysis of the V(D)J combinations, in particular by carrying out amplification steps (b) in parallel with various pairs of primers. This method is both qualitative and quantitative. Detection of the V(D)J genetic recombinations makes it possible to monitor the specific immune repertoire and to estimate the diversity thereof dynamically under various pathological conditions and following various treatments. Based on the principle of DNA amplification, this technique makes it possible to detect the immune repertoire early and with a small amount of biological material.

The measurement of these repertoires constitutes a difficult challenge in view of the large number of combinations of the V(D)J repertoire. Furthermore, each receptor consists of two chains, and thus the total repertoire for a given receptor is the product of the structural diversity of each of the chains constituting it. The numbers of possible forms of Ig or TCR receptors are estimated to be of the order of 10¹⁴ to 10¹⁸ different potential receptors. However, the evaluation of the repertoires remains an important investment for evaluating the specific immune response under physiopathological conditions such as responses to infections, to the development of cancer, to allergies, to autoimmune diseases, to immune deficiencies and to the reconstruction thereof, etc.

Firstly, it may be considered that the diversity of the immune repertoire is in relation to the organism's health. A weakly diversified repertoire can correspond to an immune deficiency, rendering the organism sensitive to various pathologies. In a certain number of medical and clinical fields, it is important to monitor variations in the repertoires. Mention may be made of situations of hereditary lymphopenias (congenital immune deficiencies) or infectious lymphopenias (AIDS, for example), or provoked lymphopenias as in the case of immune system ablations by body irradiation in the case of treatments for blood cancers (leukemias, lymphomas, etc.).

Secondly, a certain number of therapeutic trials are aimed at inducing a specific immune response. This is the case, for example, of the various antimicrobial and antitumor vaccination protocols using various routes of injection, formulations, adjuvants, various vectors, etc. In other cases, a repression of the immune response is sought, for instance to counteract autoimmune syndromes and to combat allergic manifestations.

In all these cases, the method according to the invention makes it possible, firstly, to measure the antigen receptor repertoire during the various phases of the diseases, and, secondly, to evaluate the stimulating or repressive action of molecules with the aim of deriving therefrom medicinal products that modulate the specific immune response.

Consequently, a subject of the present invention is also:
a method for the follow-up to a treatment for a pathology in which the immune repertoire is initially modified, in an individual concerned, which method is characterized:
in that it implements the method for the evaluation of the immune repertoire, as defined above, at the beginning of treatment, and
in that said evaluation method is reiterated at various phases of the treatment, and
in that the profile of the immune repertoire obtained each time is compared with that of a standard immune repertoire, in order to evaluate the response of said individual to said treatment.
a method for the measurement of the antigen receptor repertoire during the various phases of a pathology in which the immune repertoire is modified, in an individual concerned, which method is characterized:
in that it implements the method for the evaluation of the immune repertoire, as defined above, at various phases of the pathology, and
in that the profile of the immune repertoire obtained each time is compared with that of a standard immune repertoire, in order to evaluate the evolution of said pathology.

In accordance with the invention, the biological sample consists of T lymphocytes of any origin; more specifically, the biological sample can consist of thymic cells, of T lymphocytes from peripheral blood or from other lymphoid organs, or of T lymphocytes originating from other tissues, and in particular T lymphocytes derived from tumors, from inflammatory sites, or from various organs such as intestine, lung, liver, etc.

A subject of the present invention is also a kit for the quantitative evaluation of the immune repertoire of an individual, characterized in that it comprises, in addition to the usual buffers and reagents for carrying out a PCR, the primers and the probes as defined above.

The pathologies concerned are both tumors of lymphocytes and lymphocyte-related cells and any other pathology involving the immune response, such as viral diseases, autoimmune diseases, physiopathological states, immunodeficiencies, allergies, or anomalies in V(D)J recombination mechanisms.

A subject of the present invention is also primers that can be used in a method as defined above, characterized in that they are selected from the group consisting of the oligonucleotide primers corresponding to the sequences SEQ ID NO: 1-21.

A subject of the present invention is also detection probes that can be used in a method as defined above, characterized in that they are selected from the group consisting of the oligonucleotide probes of sequences SEQ ID NO: 22-37.

A subject of the present invention is also the use of the amplification primers and of the detection probes as defined above, for the quantitative evaluation of the immune repertoire of an individual.

Besides the above arrangements, the invention also comprises other arrangements, which will emerge from the description that follows, which refers to examples of implementation of the method that is the subject of the present invention and also to the attached drawings, in which:

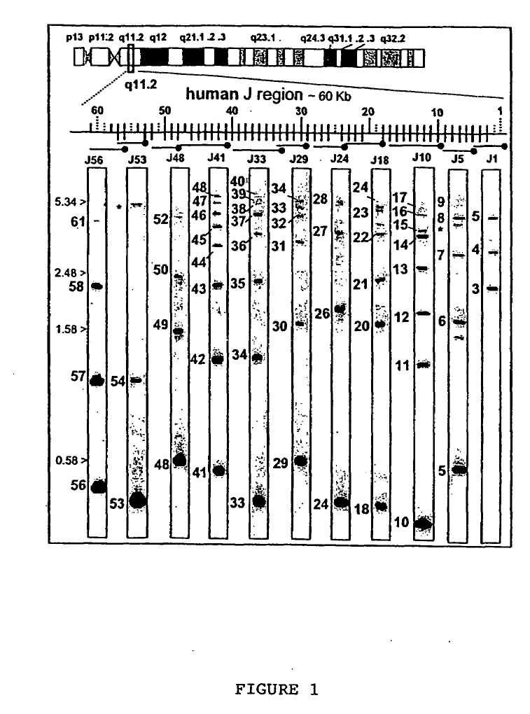

FIG. 1 represents the results of a method according to the invention (multiplex long PCR) carried out so as to evaluate the V8-Jα rearrangement in the thymus: overall analysis of the Jα region. The multiplex PCR reactions are carried out on gDNA using the primers Vα of the hV8 family (see table 1) in combination with 11 different primers Jα (see table I: SEQ ID NO: 11-21). The products are analyzed by Southern blotting using probes specific for the V8 family (SEQ ID NO: 24). Each lane corresponds to an individual PCR reaction; each band corresponding to a rearrangement with the Jα segment indicated on the left of the lane. Given the fact that PCR amplification is more efficient on small products than on large products, the bands located at the bottom of each lane are more intense. The asterisks indicate the nonspecific products determined by the distance of migration. Representation of the J region (not to scale): the 11 primers chosen are distributed over the Jα region as indicated. The dots indicate the position of the primers Jα and of the probes Jα; the corresponding lines indicate the extent of the Jαsegments detectable in each lane. The dashed lines indicate the untranscribed Jα pseudogenes;

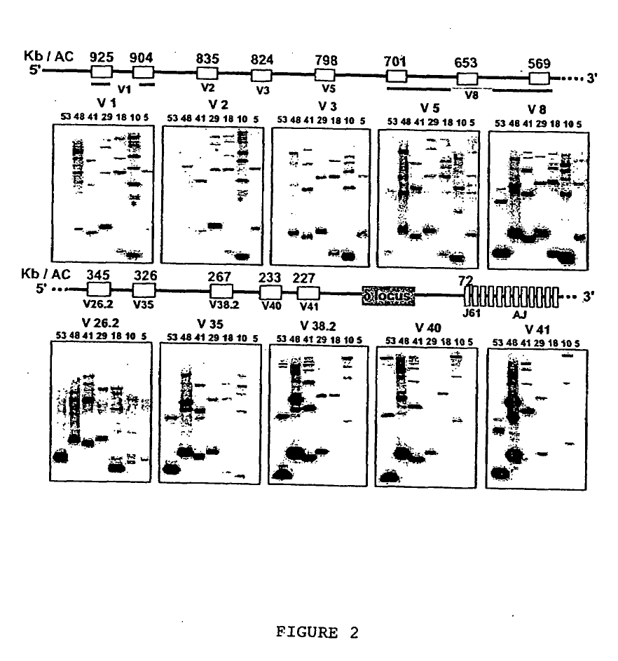

FIG. 2 illustrates the role of the location of the Vα genes in the distribution of the rearrangement with the Jα region. The V-Jα rearrangements are analyzed by multiplex PCR on DNA extracted from total thymus of 6-day-old infants. The primers specific for the Vα genes are indicated and correspond to the primers described in the table above; these primers are used with 7 primers J indicated above each lane (see also the sequences in table I above);

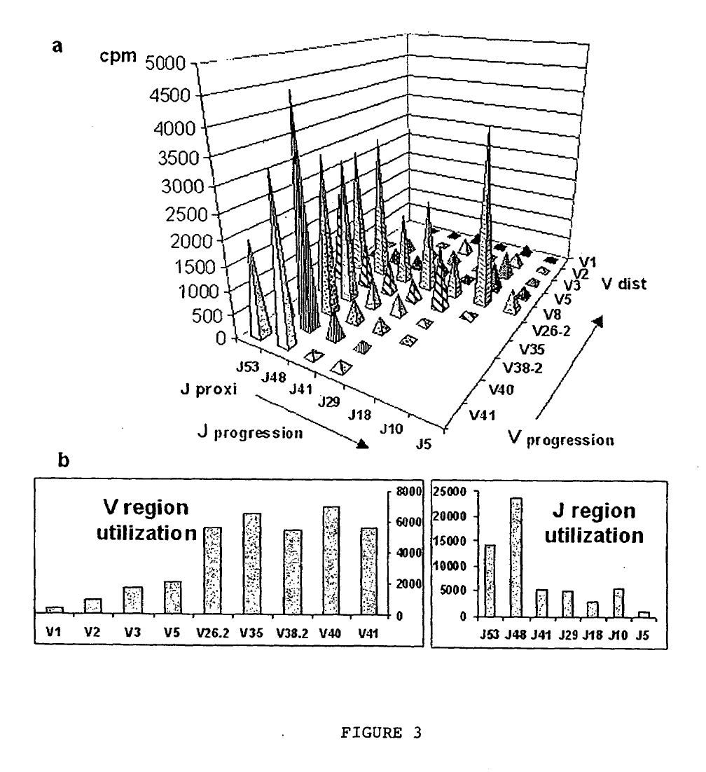

FIG. 3 illustrates a representation of the human V-Jα repertoire matrix, the rearrangement depending on the position of said genes on the locus. FIG. 3a: panel representative of the relative quantification of a V-Jα rearrangement, by integration of the signals of the PCR products of FIG. 2. This integration is carried out on the PCR products for which the PCR reaction is stopped during the exponential phase and after verification that the integration is linear. The Z-axis illustrates the relative signal intensity of the rearrangements detected; the J-axis represents the relative order of the Jα segments studied; the Vα genes studied are indicated on the V-axis. The arrows illustrate the progression tendencies of the Vα-Jα rearrangements. FIG. 3b left: analysis of the overall Vα region utilization by integration of the 7 Jα segments studied in FIG. 3a; FIG. 3b right: analysis of the overall Jα region utilization by integration of the 10 Vα genes studied in FIG. 3a;

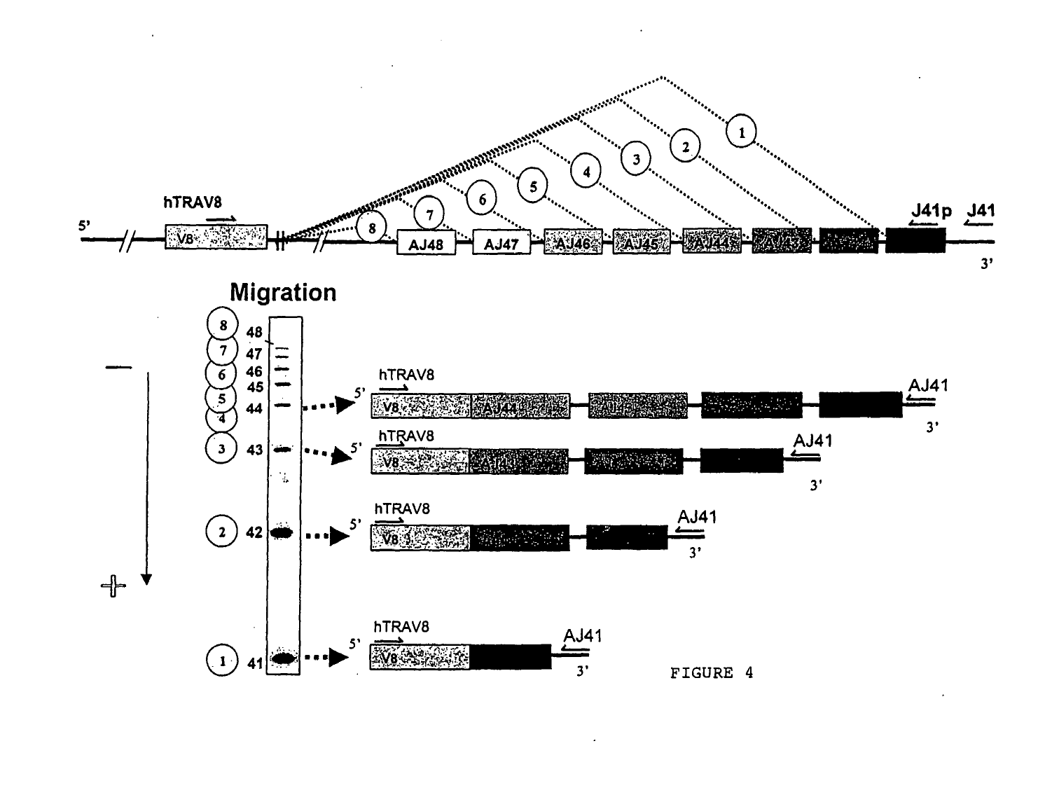

FIG. 4 illustrates the principle of the method according to the invention, applied to the detection of the V8-J recombinations: the primers used are as follows: SEQ ID NO: 5 (hTRAV8) and SEQ ID NO: 13 (hTRAJ41); the detection probe is the hAJ41p probe (SEQ ID NO: 29);

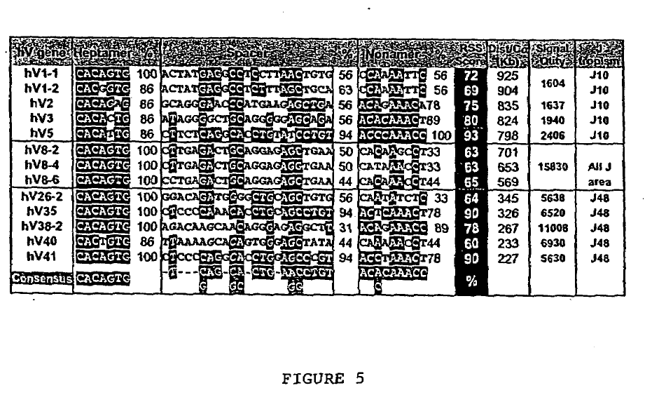

FIG. 5 represents a comparison between the RSS score of the human Vα genes, the position in the locus and the J segment principally used;

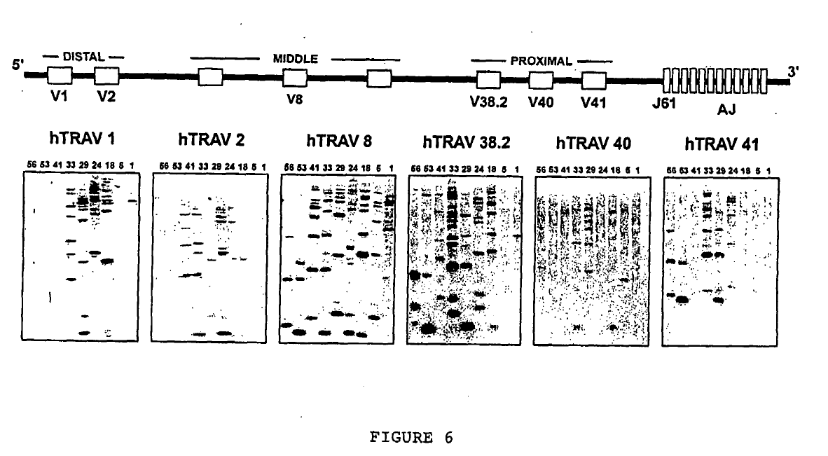

FIG. 6 represents a human TCRα chain rearrangement profile in peripheral blood lymphocytes. An example of the TCRα rearrangement profile of a donor is represented (multiplex PCR). Six Vα-specific oligonucleotides were selected and used in combination with nine Jα-specific primers (see above each lane). This analysis was carried out with three additional DNAs obtained from peripheral blood lymphocytes from normal individuals (aged 25-55) and comparable results were obtained. The multiplex PCR analyses were reproduced several times for each DNA;

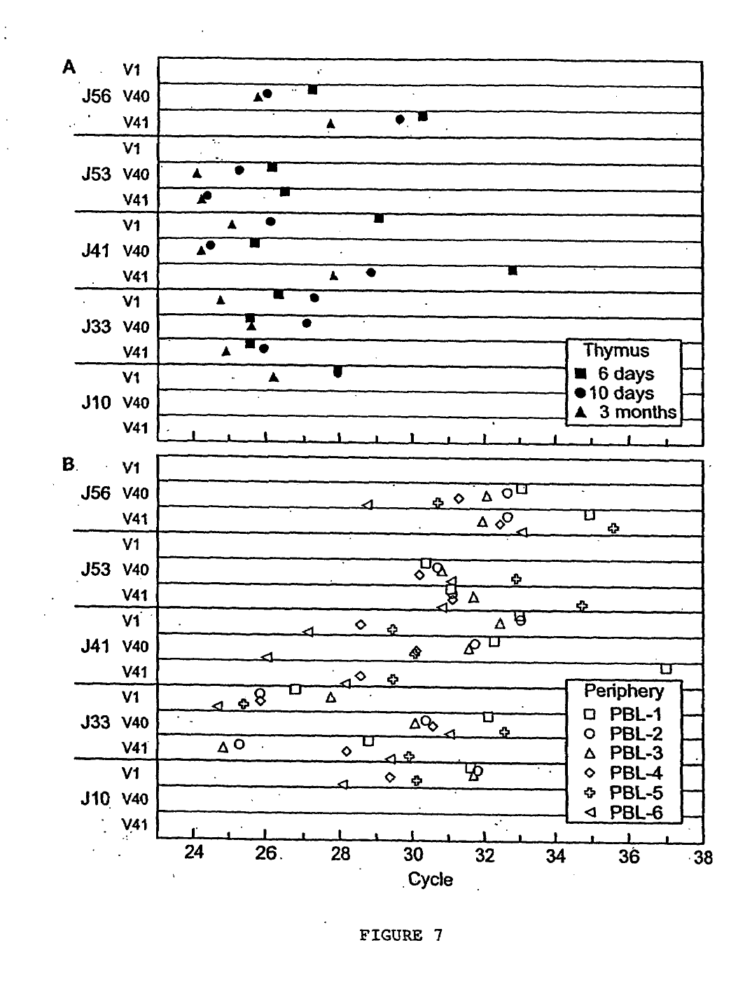

FIG. 7 illustrates the relative abundance of the specific V-Jα rearrangements in six normal individuals, determined by a quantitative genomic PCR analysis. The rearrangements selected are as follows: Vα1, 40, 41 and Jα56, 53, 41, 33, 10. For each rearrangement, the cycle during which a product is detected is indicated. Three DNAs from thymus (A) originating, respectively, from a child aged 6 days, 10 days and 3 months, and also peripheral blood lymphocytes (PBL) (B) originating from six normal individuals aged 25 to 55 were analyzed. The DNA content of each sample is normalized by amplification of the G3PDH housekeeping gene. The data presented correspond to those obtained from three different experiments which gave similar results. The results are expressed as number of cycles required for the appearance of the product related to a specific rearrangement in the various individuals. A relatively small number of cycles implies the presence of larger amounts of DNA and thus more recombinations;

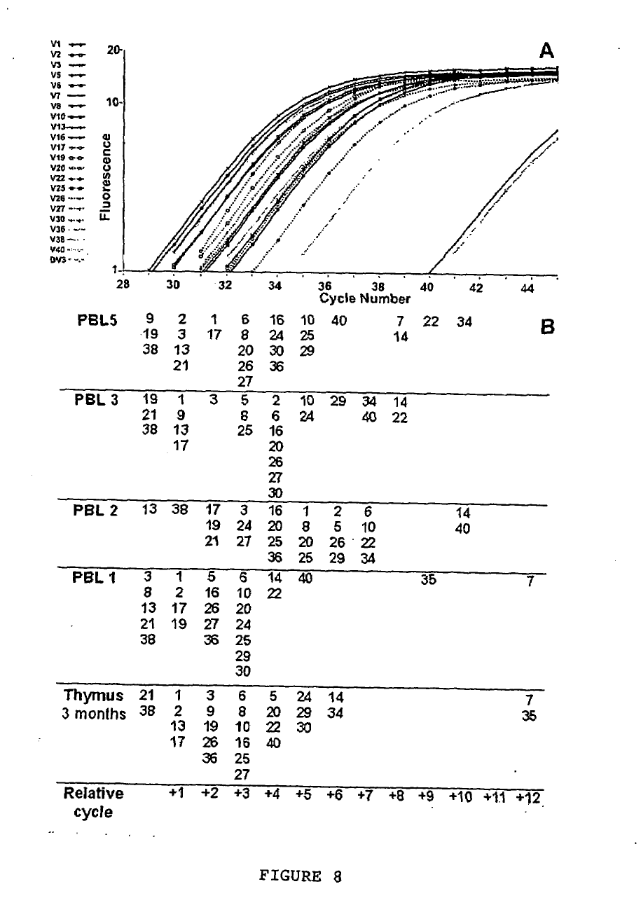

FIG. 8 represents:

in (A), the quantification of the TCRα transcripts: the cDNA extracted from peripheral blood lymphocyte samples obtained from 10 normal individuals was amplified using oligonucleotides of various ADV families in combination with a primer specific for the constant fragment of the α chain (primer AC). The amplification curve illustrated in FIG. 8(A) is representative of one of the 10 samples analyzed. The results are represented for ADV1, 3, 5, 6, 7, 8, 10, 13, 16, 17, 19, 20, 22, 25, 26, 27, 30, 36, 38, 40, and DV3. The curves correspond to the log of the fluorescence intensity as a function of the number of cycles;
in (B), the relative abundance of the ADV-AC transcripts by real-time quantitative PCR: the ADV families are indicated only by their number, in relation to the cycle during which the product is detected.

A comparison between four peripheral blood lymphocyte cDNAs and one thymus cDNA from a 3-month-old child was established.

A similar frequency of expression of the Vα families is observed in the other samples, the results obtained being reproducible.

It should be understood, however, that these samples are given only by way of illustration of the subject of the invention, of which they no way constitute a limitation.

### EXAMPLE 1

### Materials and Methods

—Nomenclature:

The nomenclatures for the TRAV (Vα) genes and the TRAJ (Jα) segments are in accordance with those of the IMGT base (http://imgt.cines.fr). The detailed map of the human TCRAD locus and of the mouse TCRAD locus are accessible on the IMGT site at the following address: http://imgt.cines.fr/textes/IMGTrepertoire/LocusGenes/locus/human/TRA/Hu_TRAmap.html.

The sequences used to establish each primer were extracted from the Genbank database, accession No. NG_{—}001332.

—Extraction of gDNA

A set of precautions must be followed in order to preserve the integrity of the genomic DNA (gDNA), in particular the size, the purity, and the absence of RNA and salt contamination. The yield from long-strand PCR amplification of DNA is conditioned by this criteria. Overall, the precautions defined for the purification of gDNA used for the Southern transfer technique (see Sambrook J., Fritsch E. F., Maniatis T. 1989. Molecular cloning. A laboratory manual. Second Edition. Cold Spring Harbor, N.Y., USA) should be observed.
Vortexing of the gDNA should be avoided, the more it is vortexed, the more it breaks.
The gDNA should be conserved in a slightly basic buffer (for example: 10 mM TRIS; pH between 7.5 and 8.5).
Abrupt pipetting should be avoided (breaking of the DNA at the tip of the pipette).
The gDNA is conserved at 4° C. in order to prevent degradation thereof.
In the event of prolonged storage, the gDNA should be placed in alcohol.

A Quiagen extraction kit is recommended: Genomic-Tip System.

http://www1.qiagen.com/Products/GenomicDnaStabilization Purification/QiagenGenomicTipSystem/

This Kit makes it possible to preserve DNA fragments of the order of 150 Kb.

The type of extraction protocol used by the Kit can be employed for working on various cell sources, for instance blood, cell culture, cells derived from cell sorting, tissues (thymus, lymph nodes, etc., list not exhaustive). In the case of tissues, before treating with the kit, 1) for tissues not very cohesive (thymus, lymph nodes, etc.), an attempt is made to individualize the cells by moderate treatment with trypsin—EDTA, 2) for highly cohesive tissues (such as muscle, for example), the tissue is reduced to powder by treatment with a pestle under cold conditions in the presence of dry ice.

—DNA Samples:
Thymus

The human genomic DNA from 3 whole thymuses (one 6-day-old female infant, two male infants 10 and 90 days old) is extracted and amplified as described in Gallagher et al. (28), the multiplex PCR and the Southern blotting are carried out as described in Mancini et al. (27).
Peripheral blood mononuclear cells (PBMCs)

The cells are separated by Ficoll density gradient. The samples originate from normal individuals aged 25 to 55. The cDNAs are extracted as described in Pernollet M. et al.

—Multiplex PCR:

Briefly, the multiplex PCR is carried out using primers located upstream of V and downstream of J (see table I above) which make it possible to amplify up to 10 kb. After each reaction, the specificity of the PCR products is verified by hybridization of internal probes V (SEQ ID NO: 22-26) and J (SEQ ID NO: 27-37) (see table II above) and by computer analysis of the migration distance (Quantity One 4.2.1 Software-Biorad, France).

The amplifications are carried out with 1.3 units per reaction of “expend high fidelity PCR system” (Roche Diagnostics, Meylan, France) under the following conditions:
initial denaturation 1-2 minutes at 94° C.
15 successive cycles comprising: a denaturation step: 5 to 30 seconds at 90-94° C., a primer hybridization step: 15-30 seconds at 58-62° C. and an elongation step: 14 to 20 minutes at 68-72° C., and
15 successive cycles comprising: a denaturation step: 5 to 30 seconds at 90-94° C., a primer hybridization step: 15-30 seconds at 58-62° C. and an elongation step: 14 to 20 minutes at 68-72° C.+15-20 additional seconds at each additional cycle, and
one final elongation cycle of 10 minutes at 72° C.

Alternatively, the conditions can also be as follows:
initial denaturation of 5 min at 94° C.,
26 successive cycles comprising a denaturation step: 30 seconds at 94° C.; a primer hybridization step: 30 seconds at 58° C. and an elongation step: 10 minutes at 72° C., and
a final elongation cycle of 10 minutes at 72° C.

The normalization of the amount of DNA in each reaction is determined by amplification of a gene not subjected to rearrangement, in the same PCR cycles. The efficiency of the primers is verified by a successive dilution of the matrix (28).

All the primers selected (see table I: SEQ ID NO: 1-21) exhibit an amplification efficiency of 98%, allowing a direct relative comparison.

The DNA fragments are separated by electrophoretic migration (agarose gel, polyacrylamide gel, microcapillary separation on an AGILENT bioanalyzer or else pulsed-field migration).

The visualization can be carried out by the following techniques, which are not limiting: i) Southern transfer onto a nylon membrane and hybridization of labeled probes (radioisotopes or fluorochromes), ii) use of a labeled base during the amplification and measurement of the incorporation thereof directly in the gel, iii) use of a DNA-labeling agent (EtBr or Cybergreen) during the amplification, or else iv) use, during the amplification, of oligonucleotides labeled with fluorochromes or other enzymatic means of visualization (avidin-biotin, peroxidase).

—Primer Design:

The primers specific for the Vα and Jα gene segments of the human TCRAD locus were selected for their sequence specificity, as specified above, using the NTI vector-8suite software, Informax. The nonspecific hybridization is verified on Blast in the site www.ensembl.org. The primers selected correspond to those illustrated in table I above.

More specifically:
for the V genes: at the genomic level, the elements corresponding to a regulatory region of promoter type before the gene, a region encoding the signal peptide or “leader” (Exon 1), an intron and a region encoding the variable region (Exon 2) and an untranslated RSS region ensuring spatial specificity of the rearrangements are found. Thus, the variable primer (V) is chosen so as to hybridize to a coding or noncoding region, but, in all cases, upstream of the RSS sequence of the V gene;
for the J genes: the primer is chosen downstream (corresponding to the 3′ of the transcribed strand) of the J gene to be studied; it can be in the coding region of the J gene or in a noncoding region between two J genes.

—Real-Time Quantitative PCR of gDNA:

The PCR reactions are carried out on a Light cycler using the FastStart® kit at one unit/reaction (Roche Diagnostics).

50 ng of DNA are used for each reaction and the amount of DNA between the samples is normalized by amplification of the G3PDH housekeeping gene.

The amplification conditions for the DNA samples are as follows: 94° C. 10 minutes, then 41 cycles (94° C. 15 sec, 67° C. 7 sec, 72° C. 7 sec).

The specificity of the single amplification product is determined by analysis of the melting curve in accordance with the manufacturer's instructions (Roche Diagnostics) and by migration on agarose gels and determination of the size of a specific rearrangement.

Each sample is analyzed in triplicate, in three different assays.

The results are expressed as number of cycles (relative abundance) for a specific rearrangement in the various thymus and peripheral blood lymphocyte DNA samples analyzed.

—Real-Time Quantitative PCR of cDNA:

The RNA from thymus (10-day-old and 3-month-old children) and from 10 peripheral blood lymphocyte samples is isolated using the RNeasy RNA isolation kit (Quiagen), according to the manufacturer's instructions.

The reverse transcription is carried out using the Superscript II RNase H⁻ kit (Life Technologies), in accordance with the protocol suggested by the manufacturer.

The cDNAs of various synthesis reactions are mixed and the same sample is used for all the PCRs.

Appropriate dilutions of each sample are selected so as to provide equivalent amounts of product by normalization with the G3PDH housekeeping gene and the CD3 gene.

The amplification conditions for the cDNA samples are as follows: 95° C. 10 min, then 47 cycles (95° C. 15 sec, 70° C. 10 sec, 72° C. 15 sec). In order to specifically amplify a maximum number of members of the Vα gene family under the same PCR conditions, the hybridization temperature was increased to more stringent conditions.

According to the manufacturer's (Roche Molecular Biochemicals) instructions, the amplification efficiency for a given PCR reaction is calculated as follows:

E=10^{-1/slope}.

The maximum possible efficiency for a PCR is E=2: each PCR product is replicated at each cycle and corresponds to a slope of −3.3 (2=10^{-1/-3.3}).

Under the experimental conditions selected, all the V-Cα PCR reactions exhibit similar amplification curve slopes from 3.67 to 3.73, indicating comparable reaction efficiencies and providing an average yield for the ADV-AC reactions of 85-87%.

The specificity of the single amplification product is established by analysis of the melting curve, by migration in agarose gels and determination of the size of a specific rearrangement and by sequencing.

The melting curves of the PCR products are determined according to the manufacturer's (Roche Diagnostics) instructions. Each sample is analyzed in triplicate, in two different assays.

### EXAMPLE 2

### Analysis of the Human J Region Compared to Its Murin Homolog (Thymus)

Multiplex PCRs (conditions of example 1) are carried out by combining primers for an individual V gene and 11 different primers downstream of AJ (J1, J5, J10, J18, J24, J29, J33, J41, J48, J53, J56) covering the J region, as represented in FIG. 1. Each column corresponds to an individual PCR reaction and each band corresponds to a rearrangement of V with a J segment, which is indicated as a number to the left of each lane.

### Determination of the Number of Potential J Segments Used in the V-J Rearrangements

Three of the six members of the human V8 family (V8.2, V8.4 and V8.6) respectively located at 701, 653 and 569 kb of the Cα gene, are amplified by the multiplex PCR technique described above, using the primers defined above.

The results represented in FIG. 1 show that the human V8 members rearrange with the J segments ranging from Jα61 to Jα3. The entire J region is therefore accessible to recombination.

### Determination of Nonfunctional Gene Segments

The nonfunctional gene segments of the TCR loci are also called “rearrangement pseudogenes”. The method according to the invention makes it possible to characterize these pseudogenes. Both in mice and in humans, certain segments have been identified as not being functional (pseudogenes).

In mice, 16 Jα genes out of 60 are pseudogenes (24, 29), whereas in humans, up until now, only the pseudogenes Jα51, 59 and 60 have been characterized (25).

As shown in FIG. 1, five additional human Jα segments are incapable of giving a rearrangement with members of the V8 family. These five Jα segments are: Jα55, Jα25, Jα19, Jα2 and Jα1. The same thing is observed with other V families (FIG. 2).

Moreover, the nonrearrangement genes Jα46, 41, 36, 29, 20, 14, 8 and 3 described in mice are functional in humans. The human Jα region contains more functional Jα segments capable of rearrangements than the mice, which implies a more diversified J repertoire.

### EXAMPLE 3

### Qualitative Analysis of Human V-J Rearrangements

Four Vα genes furthest from the Jα region (V1, V2, V3 and V5) and five Vα genes approximate to the Jα region (V26.2, V35, V38, V40, V41, located between −345 and −227 kb relative to the Cα gene) were more particularly studied.

The V8 multigene family is used as a control for use of the J region due to the fact that it is located in the middle of the locus and that it is composed of the members located at −701, −653, −569 kb of the Cα region.

Primers specific for the V genes (see table I) are used in combination with 7 primers specific for the Jα region (53, 48, 41, 29, 18, 10 and 5) (FIG. 2). To facilitate the analysis, just one V gene at a time is subjected to the rearrangement with the Jα region. The study is carried out under the multiplex PCR conditions of example 1 (see also FIG. 4).

FIG. 2 represents the profile of rearrangement of the V genes taken individually with the Jα region. It makes it possible to observe that the proximal V genes, relative to the Jα region, rearrange mainly with the most proximal 5′ J genes (V26.2, V35, V38, V40 and V41 with the Jα53, Jα48, Jα41, Jα29 segments).

These results also show that the distal V genes, relative to the Jα region, rearrange mainly with the Jα segments located at the center and in the 3′ portion of this region.

The control V8 gene makes it possible to verify that the recombination is homogeneous whatever the Jα region. FIG. 4 shows the recombinations obtained with the primers SEQ ID NO: 5 and 13 and the detection probe SEQ ID NO: 29.

The V-J rearrangements are therefore dependent on the location of the segments on the chromosomes, and each V gene rearranges with a restricted quantity of J segments.

### EXAMPLE 4

### Quantitative Analysis of Human V-J Rearrangements

The V-J rearrangements can, under the conditions of example 1, be analyzed quantitatively using a relative quantification of the intensity of each DNA band corresponding to the various rearrangements, using an imager and appropriate software such as Quantity-One (Biorad).

FIG. 3a shows a matricial representation of the human V-Jα repertoire. The Z-axis shows the integration of the relative intensity of the rearrangements detected, the J-axis represents the order of analysis of the segments and the V-axis represents the V genes studied, ranging from the proximal zone to the distal zone.

It is noted, on this figure, that the genes located between V41 and V26.2 (located at 345 kb of the TCRD locus) rearrange 3 to 7 times more than those between V5 and V1, located at 798 and 925 kb, respectively. As regards the J region, the most proximal 5′ genes rearrange 3 to 7 times more than the distal genes. Finally, the V8 multigene family combines at equal frequency with all the J gene segments, whatever their position, confirming the hypothesis that the rearrangements depend on the position of the genes on the locus.

FIG. 3b represents, for the V genes (on the left) and the J genes (on the right), the overall use of each of the segments.

### EXAMPLE 5

### TCRα Repertoire of Human Peripheral T Lymphocytes

To determine the TCRα gene recombination profiles of peripheral T lymphocytes, an approach similar to that employed for the thymus is used (see example 2).

Six genes of the Vα family were selected (TRAV), distributed in the distal region of the locus (V1 and V2), in the central region of the locus (V8) and in the proximal region of the locus (V38, V40 and V41).

Their rearrangement profiles using nine primers Jα were studied.

The analysis was carried out on four independent samples, originating from four normal individuals (25 to 55 years old).

The results obtained indicate that the recombination profiles of the TCRAD locus are remarkably similar in the various normal individuals.

TABLE IV
Data from the multiplex PCR on four DNA samples
obtained from peripheral blood lymphocytes
of normal individuals (PBL1 to PBL4)

| V/J | | 56 | 53 | 48 | 41 | 33 | 29 | 24 | 18 | 5 | 1 |
|---|---|---|---|---|---|---|---|---|---|---|---|
| V1 | PBL 1 | − | − | ND | − | + | + | + | + | − | + |
| | PBL 2 | − | − | − | + | + | + | + | + | − | + |
| | PBL 3 | − | − | ND | − | + | + | + | + | − | + |
| | PBL 4 | − | − | − | + | + | + | + | + | − | + |
| V2 | PBL 1 | − | − | ND | + | + | + | + | + | + | − |
| | PBL 2 | − | − | − | + | + | + | + | + | + | − |
| | PBL 4 | − | − | + | − | + | − | ND | − | − | + |
| V8 | PBL 1 | + | + | + | + | + | + | + | + | + | + |
| | PBL 2 | + | + | + | + | + | + | + | + | + | + |
| | PBL 3 | + | + | + | + | + | + | + | + | + | + |
| | PBL 4 | + | + | + | + | + | + | + | + | + | + |
| V38 | PBL 1 | + | + | + | + | + | + | ND | + | − | + |
| | PBL 3 | + | + | + | + | + | + | + | − | − | − |
| | PBL 4 | + | + | + | + | + | + | ND | + | − | − |
| V41 | PBL 1 | + | + | − | + | + | + | ND | − | − | − |
| | PBL 2 | + | − | ND | + | − | + | +/− | + | − | − |
| | PBL 3 | + | − | ND | + | − | +/− | +/− | + | − | − |
| | PBL 4 | + | − | ND | + | − | + | − | + | − | − |
| V40 | PBL 1 | + | + | − | + | + | + | ND | − | − | − |
| | PBL 2 | − | + | ND | + | + | +/− | + | − | − | − |
| | PBL 4 | + | + | + | + | + | +/− | ND | − | − | − |

The results obtained show that the proximity “rule” observed in the thymus also applies.

The rearrangement profile of a lymphocyte sample (among the four samples analyzed) is represented in FIG. 6.

In particular, the most proximal Vα genes (V38, V40 and V41) are mainly rearranged with the proximal Jα genes.

The V8 multigene family, located in the middle of the Vα locus, is rearranged with all the Jα segments of the locus equally.

The distal Vα genes (V1 and V2) are rearranged preferably with the central and 3′ portion and also with the closest Jα genes.

In certain individuals, discrete differences are observed in their combinatorial profiles (see Table V). For example, the rearrangements involving V1-J41, V2-J41 and V2 with the most distant J genes or, for the Vαs, the most proximal J genes, the rearrangements involving V40-J56, J29 and V41-J53, J24, are not always detected, in comparison with the thymus.

However, these differences do not affect the general combinatorial rules and confirm the similarity of the profiles between normal individuals.

—Analysis of the Frequencies of the Specific Rearrangements in Various Human Peripheral T Lymphocyte DNA Samples:

Although the recombination profiles are similar in the thymus and the peripheral lymphocytes, the multiplex PCR profiles show different rearrangement frequencies for specific combinations.

To analyze these differences more precisely, a real-time quantitative PCR carried out using gDNA was developed.

The rearrangements were analyzed on DNA obtained from peripheral lymphocytes of six normal individuals, including three of the four samples tested by multiplex PCR, and also the three thymic DNAs tested by multiplex PCR.

The rearrangements V1, V40 and V41 in combination with the proximal genes J56 and J53, the central genes J41 and J33 and the distal gene J10 were more particularly studied.

The results are illustrated in FIG. 7 and are expressed as number of cycles for the appearance of the product of each rearrangement, the first product to appear being the most abundant.

Based on these results, the following observations can be made:
Some V-Jα combinations are not detectable by the PCR analyses carried out, doubtless due to the fact that they are not very frequent (V1-J56, V1-J53, V40-J10 and V41-J10). This result confirms the combinatorial profile already observed (see FIGS. 2 and 6), which depends on the reciprocal positions of the Vα and Jα genes in the locus and implies, for all the DNAs tested, a proximal Vα-proximal Jα and distal Vα-distal Jα combination;
The rearrangements are quantitatively slightly less numerous in the cells of the periphery compared with the thymus (FIGS. 7A and 7B).

In particular, the V-Jα proximal rearrangements, such as V40-J56 or V41-J56 and V41-, V40-J53, are found in small amount in the DNA of the lymphocytes tested (8 to 64 times less), compared with what is found in the thymus.

These differences can be explained in several ways: 1) the different number of T cells between the thymus and peripheral blood; 2) the contribution of the rearrangements on the excision circles, which could be amplified in the thymus, is diluted in peripheral T cells; 3) the occurrence of secondary rearrangements in the thymus or of receptor revision events at the periphery, which could replace proximal rearrangements with the junction of more distal V-Jα segments; 4) negative selection events;
Some combinations, such as V1-J33, are favored at the periphery (high frequency=low number of cycles) in all the individuals tested.
Specific rearrangement expansions/contractions can also be identified in certain individuals: V40-J41, V1-J41, V1-J10, V41-J41, V41-J33, V40-J33.

Other rearrangements, such as V41-J41, are not found in certain individuals and probably reflect a negative selection event.

These results show that, although the recombination profile is quantitatively similar among the various thymus samples, a greater heterogeneity is observed among the peripheral T lymphocyte samples.

This divergence among individuals is linked to various events such as thymic selection, expansions of certain clonotypes induced by an immune response or homeostasis maintenance forces.
Frequency of V family transcripts in several individuals:
   prior tests (48), using a semi-quantitative analysis of the Vα gene, have shown a bias in terms of preferential expression of certain Vα segments on CD4+ or CD8+ cells.

However, a fine analysis of the prevalence of certain specific Vα families has not been established due to the absence of specific Vα reagents.

To overcome this problem, a real-time quantitative PCR analysis was developed.

Specific primers were selected (for 26 out of the 34 Vα families), which make it possible to cover approximately 77% of the families. The efficiency obtained, due to the choice of primers made, made it possible to compare the frequencies of expression of the Vα families in various individuals.

Table V represents the primers used to measure the frequency of the V family transcripts.

TABLE V

| | |
|---|---|
| Primer | Sequence 5′-3′ |

| | | |
|---|---|---|
| hTRAV 1b | GCAACATGCTGGCGGAGCACCCAC | (SEQ ID NO: 46) |
| hTRAV 2b | ATGGCTTTGCAGAGCACTCTGG | (SEQ ID NO: 47) |
| hTRAV 3c | GCCTCTGCACCCATCTCGA | (SEQ ID NO: 48) |
| hTRAV 5b | GAGGATGTGGAGCAGAGTCTTTTC | (SEQ ID NO: 49) |
| hTRAV 6 | CGGCCACCCTGACCTGCAACTATA | (SEQ ID NO: 50) |
| hTRAV 7C | GGGACCCCAGCAGGGAGACGTTGCC | (SEQ ID NO: 51) |
| hTRAV 8-1 | ATGCTCCTGTTGCTCATACCAGTG | (SEQ ID NO: 52) |
| hTRAV 9-2 | CCTGAAAGCCACGAAGGCTGATGA | (SEQ ID NO: 53) |
| hTRAV 10C | GCATCTGACGACCTTCTTGGT | (SEQ ID NO: 54) |
| hTRAV 12-b | CCATGATGCGGGGACTGGAGTTGC | (SEQ ID NO: 55) |
| hTRAV 13-1 | CATTCGTTCAAATGTGGGCGAAAA | (SEQ ID NO: 56) |
| hTRAV 14c | CAGAAGATAACTCAAACCCAACCA | (SEQ ID NO: 57) |
| hTRAV 16b | AGAGTGACTCAGCCCGAGAAG | (SEQ ID NO: 58) |
| hTRAV 17 | CCGGGCAGCAGACACTGCTTCTTA | (SEQ ID NO: 59) |
| hTRAV 19 | TCGTCGGAACTCTTTTGATGAGCA | (SEQ ID NO: 60) |
| hTRAV 20 | GTCTTGTGGCTTCAGCTTGGC | (SEQ ID NO: 61) |
| hTRAV 21 | TGCCTCGCTGGATAAATCATCAGG | (SEQ ID NO: 62) |
| hTRAV 22D | GGGAGCTCTGCTGGGGCTCTTGAG | (SEQ ID NO: 63) |
| hTRAV 24B | GCAGCTTCCCTTCCAGCAAT | (SEQ ID NO: 64) |
| hTRAV 25 | GGAGAGGACTTCACCACGTACTGC | (SEQ ID NO: 65) |
| hTRAV 26/DV7B | GGCTGGTGGCAAGAGTAACTG | (SEQ ID NO: 66) |
| hTRAV 27 | CACTGCGGCCCAGCCTGGTGATAC | (SEQ ID NO: 67) |
| hTRAV 29B | CAGCAAGTTAAGCAAAATTCACCA | (SEQ ID NO: 68) |
| hTRAV 30c | GCCGTGATCCTCCGAGAAGGGG | (SEQ ID NO: 69) |
| hTRAV 34 | TGATGATGCTACAGAAAGGTGGGG | (SEQ ID NO: 70) |
| hTRAV 35 | GGCTGGGAAGTTTGGTGATATAGTGTC | (SEQ ID NO: 71) |
| hTRAV 36/DV7 | ATGATGAAGTGTCCACAGGCT | (SEQ ID NO: 72) |
| hTRAV 38 | AGCAGCCAAATCCTTCAGTCTCAA | (SEQ ID NO: 73) |
| hTRAV 40 | AAGACAAAAACTCCCCCATTGTGAAATA | (SEQ ID NO: 74) |
| hTRDV 3 | CAGAGTTCCCCGGACCAGAC | (SEQ ID NO: 75) |

The quantitative PCR results are expressed in terms of number of cycles for the appearance of a given product for each V-Cα family.

The order of appearance of the various V-Cα products corresponds to their relative abundance in the cDNA considered in the sample, the most abundant transcript being detected first.

An amplification curve representative of a cDNA from normal peripheral T lymphocytes, obtained from 10 individuals, is represented in FIG. 8A.

The data indicate that the human Vα families are not expressed to the same degree in T lymphocytes when selection is from the thymus and from mature peripheral T lymphocytes; these data also show that the frequency of Vα expression in individuals of different species and with various types of MHC are nevertheless similar (FIG. 8B).

An ADV family expression profile can thus be identified by analyzing the frequencies of the Vα gene.

If it is considered that a maximum difference in amplification efficiency is 2% per cycle among the various V-Cα combinations, over, for example, 30 amplification cycles, this implies that there may be one cycle of difference in the measurement of the various products.

Several groups can thus be defined:
group 1: appearance at the 1st or at the 2nd cycle (high expression): Vα21, 13, 38, 19 and 17;
group 2: expression 4 to 16 less than that of group 1: Vα27, 26, 16, 5, 36, 29, 10, 20, 30, 6, 25 and

Some Vα families, such as V1, V2, V3 and V8, can be found in group 1 or group 2 depending on the individuals;
group 3: genes always weakly expressed (more than 5 cycles of difference with the gene most expressed): Vα7, 14, 22, 40, 34 and 25.

In order to evaluate the level at which the Vα expression is established, a supplementary analysis was carried out on thymus DNA. In the thymus (see FIG. 8B), the same profile as in the peripheral T lymphocytes is observed, with the exception of V40, which appears to be expressed more in the thymus.

These various levels of expression of the Vα families add additional bias in the generation of TCR repertoires and shows the advantage of the method according to the invention, in which it is the V/J pairing, as such, which is analyzed.

### BIBLIOGRAPHICAL REFERENCES

1. FINK P. J. et al., Journal of Experimental Medicine, 1978, 148, 766-775.
2. DAVIS M. M. et al., Nature, 1988, 334, 395-402.
3. SAITO H. et al., Nature, 1984, 309, 757-762.
4. GARCIA K. C. et al., Annu Rev Immunol, 1999, 17, 369-397.
5. ALLISON T. J. et al., Nature, 2001, 411, 820-824.
6. HENNECKE J. et al., Cell, 2001, 104, 1-4.
7. HAMROUNI A. et al., J Exp Med, 2003, 197, 601-614.
8. DIETRICH P. Y. et al., J Immunol, 2003, 170, 5103-5109.
9. BASSING C. H. et al., Cell, 2002, 109, suppl: S45-55.
10. CHIEN Y. H. et al., Nature, 1984, 309, 322-326.
11. SLECKMAN B. P. et al., Immunol Rev, 1998, 165, 121-130.
12. CAPONE M. et al., Proc Natl Acad Sci USA, 1998, 95, 12522-12527.
13. VON BOEHMER H. et al., Curr Opin Immunol, 1999, 11, 135-142.
14. GELLERT M., Adv Immunol, 1997, 64, 39-64.
15. OETTINGER M. A., Science, 1990, 248, 1517-1523.
16. GELLERT M., Annu Rev Biochem, 2002, 71, 101-132.
17. McBLANE J. F. et al., Cell, 1995, 83, 387-395.
18. VAN GENT D. C. et al., Cell, 1996, 85, 107-113.
19. HESSE J. E. et al., Genes Dev, 1989, 3, 1053-1061.
20. COWELL L. G. et al., J Exp Med, 2003, 197, 207-220.
21. CABANIOLS J. P. et al., J Exp Med, 2001, 194, 1385-1390.
22. SAITO T. et al., J Exp Med, 1988, 168, 1003-1020.
23. CASROUGE A. et al., J Immunol, 2000, 164, 5782-5787.
24. PASQUAL N. et al., J Exp Med, 2002, 196, 1163-1173.
25. KOOP B. F. et al., Genomics, 1994, 19, 478-493.
26. GLUSMAN G. et al., Immunity, 2001, 15, 337-349.
27. MANCINI S. J. et al., J Immunol, 2001, 167, 4485-4493.
28. GALLAGHER M. et al., J Immunol, 2001, 167, 1447-1453.
29. GAHERY-SEGARD H. et al., Immunogenetics, 1996, 44, 298-305.
30. KRANGEL M. S., Nat Immunol, 2003, 4, 624-630.
31. YANCOPOULOS G. D. et al., Cell, 1986, 44, 251-259.
32. MOSTOSLAVSKY R. et al., Nat Immunol, 2003, 4, 603-606.
33. STRAHL B. D. et al., Nature, 2000, 403, 41-45.
34. McBLANE et al., Curr Biol, 2000, 10, 483-486.
35. HSIEH C. L. et al., Embo J, 1992, 11, 315-325.
36. SPICUGLIA S. et al., Mol Cell, 2002, 10, 1479-1487.
37. HUANG C. et al., J Immunol, 2001, 166, 2597-2601.
38. WANG F. et al., Proc Natl Acad Sci USA, 1998, 95, 11834-11839.
39. DAVODEAU F. et al., Embo J, 2001, 20, 4717-4729.
40. McMURRY M. T. et al., Science, 2000, 287, 495-498.
41. MAUVIEUX L. et al., Eur J Immunol, 2001, 31, 2080-2086.
42. VILLEY I. et al., Immunity, 1996, 5, 331-342.
43. ARSTILA T. P. et al., Science, 1999, 286, 958-961.
44. BASSING C. H. et al., Nature, 2000, 405, 583-586.
45. LEE A. I. et al., PloS Biol 1: E1, 2003.
46. HODGES E. et al., J Clin Pathol, 2003, 56, 1-11.
47. PERNOLLET M. et al., Clin. Exp. Immunol., 2002, 130, 518-525.
48. GULWANI-AKOLKAR B. et al., J. Immunol., 1995, 154, 3843-3851.

1. A method for the quantitative evaluation of a rearrangement or of a targeted genetic recombination of an individual, which method comprises at least:
(a) the extraction of human genomic DNA from a biological sample,
(b) the amplification of a segment of said genomic DNA, between a few hundred base pairs and several tens of kb in size, by multiplex PCR, in the presence:
of one or more pairs of primers, selected so as to correspond to the following characteristics:
at least one of said primers of one of said pairs of primers hybridizes upstream and/or at the 5′ end of a Vx gene to be amplified, which may be involved in said genetic rearrangement;
at least the other of said primers of one of said pairs of primers hybridizes downstream and/or at the 3′ end of a Jy gene to be amplified, which may be involved in said genetic rearrangement;
and of a DNA polymerase or a mixture of DNA polymerases for amplifying genomic DNA segments between a few hundred base pairs and several tens of kb in size, preferably greater than 10 kb in size, and having a correction activity that makes it possible to substantially improve the elongation;
said amplification comprising, in addition to the initial denaturation step, cycles of denaturation, hybridization and elongation, in which the elongation steps are carried out at least for 10 minutes at 68° C.-72° C.;
c) the separation of the gDNA fragments amplified, and
d) the detection of the rearranged or recombined segments.

2. The method for the quantitative evaluation of the immune repertoire of an individual by genetic rearrangement as claimed in claim 1, comprising:
a) the extraction of human genomic DNA from a biological sample,
b) the amplification of a segment of said genomic DNA, between a few hundred base pairs and several tens of kb in size, by multiplex long PCR, in the presence:
of one or more pairs of primers, selected so as to correspond to the following characteristics:
at least one of said primers of one of said pairs of primers, called primer V, hybridizes specifically with a region located upstream of the RSS sequence of a Vx gene to be amplified, corresponding to a V segment of the variable domain of the α chain of a T-cell receptor (TCRAD);
at least one of said primers of one of said pairs of primers, called primer J, hybridizes specifically with a region located downstream of the RSS sequence of a Jy gene to be amplified, with the 3′ end of said Jy gene to be amplified or in said Jy gene to be amplified, corresponding to a J segment of the α chain of a T-cell receptor;
and of a DNA polymerase or a mixture of DNA polymerases for amplifying genomic DNA segments between a few hundred base pairs and several tens of kb in size, and having a correction activity that makes it possible to substantially improve the elongation;
said amplification comprising, in addition to the initial denaturation step, cycles of denaturation, hybridization and elongation, in which the elongation steps are carried out at least for 10 minutes at 68° C.-72° C.;
(c) the separation of the gDNA fragments amplified, and
(d) the detection of the recombined V(D)J segments.

3. The method as claimed in claim 1, wherein in the amplification step (b), the selection of the primers is carried out:
by systematic analysis of the entire locus concerned, and in particular of the human TCRAD locus, using a suitable software,
selection of the primers whose 3′OH end is complementary only to the region of interest,
elimination of the primers forming autodimers or stable hairpins, in particular by analysis with a suitable software, and
elimination of the pairs of primers which form hybrids with one another.

4. The method as claimed in claim 3, wherein the primers V and J of the pairs of primers V/J are selected from the group consisting of the primers of sequences SEQ ID NO: 1-21.

5. The method as claimed in claim 1, wherein the amplification step (b) advantageously uses additional primers for amplifying, in addition, at least one of the following segments: D segments, V segments and J segments of the TCR β, γ, δ chains and, optionally, segments of the immunoglobulin chains.

6. The method as claimed in claim 1, wherein in the amplification step (b), the multiplex long PCR (LPCR) reaction is carried out after purification of the DNA, or directly on a cell lysate.

7. The method as claimed in claim 1 wherein in the amplification step (b), the elongation steps are incremented by 15-20 seconds per additional elongation cycle.

8. The method as claimed in claim 1 wherein step (c) consisting of separation of the amplified DNA fragments is carried out by electrophoretic migration on a gel, preferably pulsed-field migration.

9. The method as claimed in claim 1 wherein step (c) consisting of separation of the amplified DNA fragments is carried out by microcapillary separation.

10. The method as claimed in claim 1 wherein the detection step (d) can advantageously be carried out by Southern transfer of the amplified products onto nylon membranes, followed by visualization after hybridization with one or more nucleotide probes labeled with a radioactive isotope or a fluorochrome.

11. The method as claimed in claim 10, wherein the probes are advantageously selected from the group consisting of the sequences SEQ ID NO: 22-37.

12. The method as claimed in claim 1 wherein the detection step (d) can advantageously be carried out by using a labeled base (labeled with a radioactive isotope or a fluorochrome) during the amplification, and then by measuring the incorporation thereof directly in the gel.

13. The method as claimed in claim 1 wherein the detection step (d) can advantageously be carried out by using a DNA-labeling agent during the migration, and detecting after excitation in the UV range or at another appropriate wavelength.

14. The method as claimed in claim 1 wherein the detection step (d) can advantageously be carried out by using primers labeled with fluorochromes or other enzymatic revealing means during the amplification.

15. A method for the follow-up to a treatment for a pathology in which the immune repertoire is initially modified, in an individual in need thereof, which method comprises:
implementing the method for the evaluation of the immune repertoire, as claimed in claim 1, at the beginning of treatment,
reiterating said evaluation method at various phases of the treatment, and
comparing the profile of the immune repertoire obtained each time with that of a standard immune repertoire, in order to evaluate the response of said individual to said treatment.

16. A method for the measurement of the antigen receptor repertoire during the various phases of a pathology in which the immune repertoire is modified, in an individual in need thereof, which method comprises:
implementing the method for the evaluation of the immune repertoire, as claimed in claim 1, at various phases of the pathology, and
comparing the profile of the immune repertoire obtained each time with that of a standard immune repertoire, in order to evaluate the evolution of said pathology.

17. The method as claimed in claim 1 wherein the biological sample consists of T lymphocytes of any origin.

18. The method as claimed in claim 17, wherein said T lymphocytes are selected from the group consisting of thymic cells, of T lymphocytes from peripheral blood, of T lymphocytes from other lymphoid organs, of T lymphocytes from various organs and of T lymphocytes derived from tumors or from inflammatory sites.

19. A kit for the quantitative evaluation of the immune repertoire of an individual comprising, in addition to the usual buffers and reagents for carrying out a PCR, primers and probes corresponding to the sequences SEQ ID NO: 1-37.

20. A primer selected from the group consisting of the oligonucleotide primers corresponding to the sequences SEQ ID NO: 1-21.

21. A detection probe selected from the group consisting of the oligonucleotide probes of sequences SEQ ID NO: 22-37.

22. (canceled)
